(19) United States
(12) Patent Application Publication
(10) Pub. No.: US 2004/0029134 A1
(43) Pub. Date: Feb. 12, 2004

(54) P53-associated Parkin-like cytoplasmic protein, and related compositions and methods

(76) Inventors: Wei Gu, Emerson, NJ (US); Anatoly Nikolaev, New York, NY (US)

Correspondence Address: John P. White, Cooper & Dunham, LLP, 1185 Avenue of the Americas, New York, NY, 10036, US

(21) Appl. No.: 10/313,203

(22) Filed: Dec. 6, 2002

(51) Int. Cl. C12Q 1/68; G01N 33/53; C07H 21/04; C07K 14/47; C12P 21/02; C12N 5/06

(52) U.S. Cl. 435/6; 435/69.1; 435/320.1; 435/325; 530/350; 536/23.2

(57) ABSTRACT

This invention provides an isolated p53-associated Parkin-like cytoplasmic protein (“Parc”) having use, for example, as an anti-cancer target. This invention also provides related recombinant proteins and nucleic acids such as vectors and anti-sense molecules. Further provided are anti-Parc antibodies, protein and antibody production methods, Parc-based screening assays, methods and compositions for decreasing Parc expression and treating subjects, diagnostic methods, and related kits.

This invention was made with funding from the National Cancer Institute under Grant No. CA 85533. Accordingly, the United States Government has certain rights in this invention.

Throughout this application, various publications are referenced. Full bibliographic citations for these publications are found at the end of the specification immediately preceding the claims. The disclosures of these publications in their entirety are hereby incorporated by reference into this application.

### BACKGROUND OF THE INVENTION

The p53 protein, acting as a bona fide tumor suppressor, can induce cell growth arrest, apoptosis, and aging/cell senescence in response to various types of stress (Vogelstein et al., 2000; Sharpless and DePinho, 2002). p53 mutations have been well documented in more than half of all human tumors (Hollstein et al., 1999) and in the cells that retain wild-type p53, other defects in the p53 pathway also play an important role in tumorigenesis.

p53 promotes tumor suppression through its ability to bind specific DNA sequences and function as a transcription factor (El-Deiry et al., 1992). The importance of p53-mediated transcriptional activation is underscored by the fact that the vast majority of tumor-associated p53 mutations occur within the domain responsible for sequence-specific DNA binding (Hollstein et al., 1999).

Tight regulation of p53 is essential for maintaining normal cell growth and for its effect on tumorigenesis. Wild type p53 is a short-lived protein that is maintained as a latent form in unstressed cells. The precise mechanism by which p53 is activated by cellular stress is not completely understood. Although both post-translation modifications (phosphorylation and acetylation) and stabilization (ubiquitination) of p53 are well accepted as key events in the p53-mediated stress response, subcellular localization also appears to play a critical role in the regulation of p53 function (Vousden et al., 2002; Jimenez et al., 1999). p53 is diffusely distributed in normal unstressed cells and, in response to DNA damage and other types of stress, p53 translocates to the nucleus where it activates endogenous target genes. Thus, nuclear localization of p53 is essential for its function as a transcription factor. Indeed, wild-type p53 is functionally inactivated by abnormal cytoplasmic sequestration in many tumor types including inflammatory breast carcinoma, undifferentiated neuroblastoma, colorectal carcinoma, and retinoblastoma; constitutive cytoplasmic localization of p53 in these tumors has been linked with poor response to chemotherapy, tumor metastasis, and poor long-term patient survival (Moll et al., 1992, 1996; Bosari et al., 1995; Sun et al., 1992; Schlamp et al., 1997; Ueda et al., 1995).

The p53 protein contains three lysine-rich nuclear localization signals (NLSs) in the C-terminus (Liang et al., 1998; Shaulsky et al., 1990). NLS I (a.a. 305-322) has been implicated as the primary NLS, and NLS II (a.a. 369-375) and NLS III (a.a. 379-384) appear to increase the efficiency of nuclear import mediated by NLS I (Shaulsky et al., 1990). Many NLS-containing transcriptional factors, such as c-Myc, Max and c-Fos, are exclusively present in nuclei. However, since subcellular localization of the p53 protein is a dynamic process, and the levels of p53 distributed in the cytoplasm and nucleus vary with different cell types and cellular conditions (Jimenez et al., 1999), cellular factors/pathways that specifically regulate both nuclear import and export of p53 are likely to exist.

A number of studies have shown that nuclear export of p53 is specifically regulated by Mdm2 (Tao and Levine, 1999; Geyer et al., 2000; Boyd et al., 2000). p53 contains a leucine-rich, rev-like nuclear export signal (NES) at the C-terminus (Stommel et al., 1999; Freedman and Levine, 1998; Middeler et al., 1997), and recently, a second NES in the p53 transactivation domain has also been identified (Zhang and Xiong, 2001). Mdm2 can significantly enhance nuclear export of p53 through the C-terminal NES (Lohrum et al., 2001; Gu et al., 2001). In addition to its p53-binding domain, the ubiquitin ligase activity of Mdm2 is also critical for its ability to promote nuclear export of p53 (Geyer et al., 2000; Boyd et al., 2000). Notably, the C-terminal lysine residues of p53 that serve as both acetylation sites (Li et al., 2002b) and the sites of Mdm2-mediated ubiquitination are also required for the enhancement of p53 nuclear export by Mdm2 (Lohrum et al., 2001; Gu et al., 2001).

The mechanism by which p53 is diffusely distributed in normal unstressed cells remains unknown. Since the Mdm2 protein is often undetectable in unstressed cells, cellular factors other than Mdm2 are likely involved in retaining the p53 protein in the cytoplasm under these conditions. A number of proteins have been proposed to serve as cytoplasmic anchor proteins to block nuclear localization of p53, including ribosomal proteins (Fontoura et al., 1997; Abou Elela and Nazar, 1997), Hsc70 (Gannon and Lane, 1991), vimentin (Klotzsche et al., 1998), tubulin (Giannakakou et al., 2000), and F-actin (Metcalfe et al., 1999). As each of these identified proteins is highly abundant, and the specificity of their interactions with p53 needs further verification, none of them has received wide acceptation as the bona fide cytoplasmic anchor of p53. Nevertheless, these studies indicate that a fraction of p53 resides in the cytoplasm and the levels of cytoplasmic p53 proteins may be regulated by cytoplasmic factors.

Again, to date, no protein has been identified as a cytoplasmic anchor for p53.

### SUMMARY OF THE INVENTION

This invention provides an isolated p53-associated Parkin-like cytoplasmic protein (“Parc”). This invention also provides a recombinant protein comprising the N-terminal 770 residues of the amino acid sequence set forth in FIG. 14.

This invention further provides isolated nucleic acids encoding each of the instant proteins. Further provided are an expression vector comprising any of the instant nucleic acids, and a host vector system comprising a cell having therein the instant expression vector.

This invention further provides a method for producing a protein comprising culturing the instant host vector system under conditions permitting the expression of the protein encoded by the expression vector therein, and recovering the protein so expressed.

This invention provides a nucleic acid which hybridizes to at least a portion of an RNA encoding Parc. This invention also provides antibodies, each of which binds to one of the instant proteins.

This invention further provides a method for making an antibody which binds to Parc comprising the steps of (a) introducing Parc to a mammal under conditions which permit the generation of antibodies to an antigen; and (b) after a suitable period of time, recovering antibodies generated in the mammal which bind to Parc.

This invention provides a method for determining whether an agent inhibits binding between p53 and Parc comprising

(a) contacting the agent with p53 and Parc under conditions which, in the absence of the agent, permit the formation of a complex between p53 and Parc;

(b) determining the amount of complex formed in step (a) between p53 and Parc; and

(c) comparing the amount of complex determined in step (b) with the amount of complex formed in the absence of the agent, wherein if the amount of complex formed in step (a) is less than the amount formed in the absence of the agent, the agent inhibits binding between p53 and Parc.

This invention also provides a method for determining whether an agent inhibits binding between p53 and the p53-binding portion of Parc comprising

(a) contacting the agent with p53 and the p53-binding portion of Parc under conditions which, in the absence of the agent, permit the formation of a complex between p53 and the p53-binding portion of Parc;

(b) determining the amount of complex formed in step (a) between p53 and the p53-binding portion of Parc; and

(c) comparing the amount of complex determined in step (b) with the amount of complex formed in the absence of the agent, wherein if the amount of complex formed in step (a) is less than the amount formed in the absence of the agent, the agent inhibits binding between p53 and the p53-binding portion of Parc.

This invention further provides a method for determining whether an agent inhibits binding between p53 and Parc in a cell comprising

(a) contacting the agent with the cell under suitable conditions;

(b) determining the amount of complex formed in the cell between Parc and p53; and

(c) comparing the amount of complex determined in step (b) with the amount of complex formed in the absence of the agent, wherein if the amount of complex formed in step (a) is less than the amount formed in the absence of the agent, the agent inhibits binding between p53 and Parc in the cell.

This invention further provides a method for determining whether an agent inhibits the expression of Parc in a cell comprising

(a) contacting the agent with the cell under suitable conditions;

(b) determining the amount of Parc expressed in the cell; and

(c) comparing the amount of Parc expression determined in step (b) with the amount of Parc expression in the cell in the absence of the agent, wherein if the amount of Parc expression determined in step (b) is less than the amount of Parc expression in the absence of the agent, the agent inhibits expression of Parc in the cell.

This invention also provides a method for decreasing the amount of Parc in a cell comprising introducing into the cell an agent that specifically inhibits the expression of Parc in the cell, thereby decreasing the amount of Parc in the cell.

This invention further provides a method for treating a subject afflicted with cancer comprising administering to the subject a therapeutically effective amount of an agent that specifically inhibits the expression of Parc in the subject's cells, thereby treating the subject.

This invention provides a composition comprising a pharmaceutically acceptable carrier and an agent that specifically inhibits the expression of Parc in a cell.

This invention also provides a method for determining whether a cell is cancerous comprising determining the amount of Parc in the cell and comparing the amount so determined to the amount of Parc present in a cell either known to be cancerous or known not to be cancerous, thereby determining whether the cell is cancerous.

This invention further provides a kit for determining the amount of Parc present in a sample comprising a detectably labeled agent which binds to Parc, and instructions for use.

Finally, this invention provides a kit for determining the amount of Parc-encoding nucleic acid present in a sample comprising a nucleic acid capable of hybridizing to Parc-encoding nucleic acid, and instructions for use.

### BRIEF DESCRIPTION OF THE FIGURES

FIGS. 1A-1D Identification of p270/Parc as a novel cytoplasmic p53-interacting protein. (A) Schematic representation of the Flag-p53(175) protein used for affinity purification of p53-interacting proteins. The arginine residue of the DNA-binding domain is replaced with histidine (aa 175). (B) Collodial-blue staining of the protein marker (lane 1), a control eluate from M2 beads loaded with parental H1299 cytoplasmic cell extract (lane 2) and affinity purified Flag-p53(175) complexes from a cytoplasmic extract of the Flag-p53(175)/H1299 stable cell line (lane 3). Specific p53-interacting protein bands were peptide sequenced by mass spectrometry, and the p270/Parc peptide sequences are presented. (C) p270/Parc is present in cytoplasmic, but not nuclear, p53-containing complexes. Western blot analysis of M2 immunoprecipitates from parental H1299 cytoplasmic extract (lane 1), Flag-p53(175)/H1299 cytoplasmic (lane 2) and nuclear (lane 3) extracts by immunoblotting with p270/Parc-specific antibody (upper) or p53-specific DO-1 antibody (lower). (D) p270/Parc mRNA is ubiquitously expressed. A multiple tissue Northern filter was hybridized with p270/Parc (upper) or actin (lower) cDNA probes.

FIGS. 2A-2E Parc contains the Ring-IBR-Ring motif and a C-terminal cullin homology domain (CCH). (A) Schematic representation of the Parc and Parkin polypepetides. (B) The amino acid sequence of Parc, with the CCH, RING and IBR domains highlighted. (C) An alignment of the CCH domain of Parc with those of human cullin1 (hCul1) and cullin2 (hCul2), C. elegans cullin1 (cCul1) and cullin2 (cCul2), and yeast cullinA (yCulA). Homologous amino acid residues are highlighted. (D) An alignment of the IBR domain of Parc with those of Parkin, human ariadne (hAriadne) and ariadne2 (hAriadne2), and D. melanogaster ariadne (dAriadne) and ariadne2 (dAriadne2). Homologous amino acid residues are highlighted. (E) An alignment of the RING domains of Parc (RING1, RING2) with those of Parkin (hParkinR1, hParkinR2), human Ariadne (hAriadne), human BRCA1 (hBRCA1) and human PML (hPML1) proteins. Homologous amino acid residues are highlighted.

FIGS. 3A-3G The interaction between p53 and Parc in vitro and in vivo. (A) Direct interactions of Parc with GST-p53. The wild-type GST-p53 full-length protein (GST-p53) (lane 4), the mutant GST-p53 full-length protein (GST-p53(175)) (lane 3), the N-terminus of p53 protein (1-73)(lane 7), the middle part of p53 (100-290)(lane 8), the C-terminus of p53 (290-393) (lane 9), or GST alone (lanes 2 and 5) were used in a GST pull-down assay with in vitro translated 35S-labeled full-length Parc. (B) The N-terminus of Parc directly interacts with GST-p53. The GST-p53 full-length protein (GST-p53) (lanes 3, 6, and 9) or GST alone (lanes 2, 5, and 8) were used in a pull-down assay with the in vitro translated 35S-labeled N-terminus of Parc (1-770) (lanes 1-3), the middle part of Parc (770-1460), (lanes 4-6), and the C-terminus of Parc (1460-2517)(lanes 7-9). (C) Co-immunoprecipitation of p53 with Parc from U2OS cells. Western blot analysis of indicated whole cell extract (WCE) (lane 1), and immunoprecipitates with a Parc-specific antibody (lane 3) or preimmune serum (lane 2) by anti-p53 monoclonal antibody DO-1 (lower) or anti-Parc antibody (upper). (D) Co-immunoprecipitation of Parc with p53 from U2OS cells. Western blot analysis of whole cell extract (WCE) (lane 1), or immunoprecipitates with anti-p53 monoclonal antibody DO-1 (lane 3) or control immunoprecipitates with anti-Ras antibody (lane 2) by a Parc-specific antibody. (E) Endogenous p53 was co-depleted with endogenous Parc from U2OS cytoplasmic extracts by anti-Parc antibody. U2OS cytoplasmic extracts were incubated with preimmune serum or α-Parc antibody in the presence of protein A/G beads. Pre-immune and α-Parc immunoprecipitation flow-throughs (lanes 2 and 3) and U2OS cytoplasmic extract (lane 1) were resolved in 8% SDS PAGE and immunoblotted with anti-Parc (upper) or anti-p53 monoclonal antibody DO-1 (middle) or actin antibody (AC-15, bottom). (F) Endogenous Parc is found in cytoplasmic, but not nuclear, extracts of U2OS cells. Western blot analysis of cytoplasmic (lane 1) and nuclear (lane 2) extracts from U2OS cells with Parc-specific (upper) or p53-specific (DO-1) antibodies. (G) Parc forms a ˜1 Mda complex with p53 in the cytoplasm of U2OS cells. Chromatographic fractions (lanes 2-11) generated by the gel filtration (Superose 6, SMART system) of Flag-Parc-containing complexes (lane 1) from the cytoplasmic extract of Flag-Parc/U2OS stable cells were immunoblotted with anti-Parc (upper) and anti-p53 (DO-1) (lower) antibodies.

FIGS. 4A-4G Parc is a cytoplasmic ubiquitin ligase. (A) Ectopically expressed Parc is diffusely localized in the cytoplasm of Flag-Parc-transfected H1299 cells. These cells were stained with anti-Flag monoclonal antibody to detect Parc. (B) Self-ubiquitination of Parc in vitro. In vitro translated 35S labeled Parc was incubated with E1, E2 (GST-UbCH7) and His-ubiquitin. (C) Ubiquitination of Parc in vivo. Immunoprecipitations from H1299 cells transfected with HA-Ub (lane 1), Flag-Parc (lane 2) or both (lane 3) by the M2 (Flag) antibody, were immunoblotted with anti-HA monoclonal antibody. (D) Parc does not ubiquitinate p53 effectively in vivo. M2 immunoprecipitations from H1299 cells co-transfected with Flag-ubiquitin and either p53 alone (lane 1) or p53 and mdm2 (lane 2) or with p53 and Parc (lane 3) were immunoblotted with anti-p53 monoclonal antibody (DO-1). (E) Parc does not ubiquitinate p53 effectively in vitro. In vitro ubiquitination reactions containing GST-p53 incubated either alone (lane 1), with GST-mdm2/GST-UbcH5 (lane 2), or Flag-Parc/GST-UbCH7 (lane 3) were immunoblotted with anti-p53 monoclonal antibody (DO-1). Mammalian E1 was included in all three reactions (lanes 1-3). (F) Parc does not promote degradation of wild-type p53. H1299 cell extracts transfected with CMV-p53/CMV-GFP (lane 1), or CMV-53/CMV-GFP and CMV-mdm2 (lane 2), or CMV-p53/CMV-GFP and CMV-Parc (lane 3) were immunoblotted with anti-p53 antibody (DO-1) (upper) or anti-GFP monoclonal antibody (lower). (G) Parc does not promote degradation of the p53-mNLS mutant (K319A, K320A, K321A). H1299 transfections and immunoblotting were done as described in (F).

FIGS. 5A-5D RNAi ablation of endogenous Parc induces nuclear localization of p53 and p53-dependent apoptosis. (A) RNAi ablation of endogenous Parc expression in U2OS and H1299 cells. Whole cell extracts from Parc-RNAi or control-RNAi treated cells (U2OS and H1299), were immunoblotted with anti-Parc, anti-p53 (DO-1), anti-p21 (C19) and anti-actin (AC-15) antibodies. (B) Quantitation of subcellular p53 localization in U2OS cells transfected with Parc-RNAi or control oligonucleotide. The data represent the average of three experiments with standard deviations indicated. (C) p53-dependent induction of apoptotic response by Parc RNAi ablation. U2OS and H1299 cells transfected with either Parc-RNAi and control oligonucleotides were analysed for apoptotic cells (sub-G1) according to DNA content (PI staining). (D) Subcellular localization of p53 in U2OS cells transfected with Parc-RNAi or control oligonucleotides. The transfected cells were immunostained with anti-p53 antibody (1801) (visualized by fluorescence) and counterstained with DAPI to visualize the nuclei.

FIGS. 6A-6C Overexpression of Parc promotes cytoplasmic sequestration of ectopic p53. (A) Subcellular localization of ectopic p53 in H1299 cells transfected with CMV-p53 alone or with CMV-p53 and CMV-Parc. The transfected cells were immunostained with anti-p53 antibody (visualized by fluorescence) and counterstained with DAPI to visualize the nuclei. (B) Subcellular localization of ectopic Myc in H1299 cells transfected with CMV-Flag-Myc alone or CMV-Flag-Myc and CMV-Parc. The transfected cells were immunostained with anti-Flag monoclonal antibody (M2) (visualized by fluorescence) and counterstained with DAPI to visualize the nuclei. (C) The Ring-IBR-Ring domain of Parc is not required for Parc-mediated cytoplasmic sequestration of ectopic p53. H1299 cells were transfected with CMV-p53 alone, with CMV-p53 and CMV-Parc (full-length), or with CMV-p53 and various CMV-Parc deletion constructs. The transfected cells were immunostained as described in (A) and the p53-positive cells were quantitated for subcellular p53 localization.

FIGS. 7A-7E RNAi-mediated reduction of Parc induces p21 activation and p53 nuclear localization in neuroblastoma cells. (A) Parc is highly expressed in neuroblastoma cell lines. Whole cell extracts of primary fibroblast (IMR90) (lane 1) and neuroblastoma cell lines (IMR32, KCNR, SK-N-AS, LAN-5) (lanes 2-5) were immunoblotted with anti-Parc antibody (upper) and anti-actin antibody (lower). (B) Parc expression levels in human cerebral cortex, glioma and neuroblastoma cell lines. Western blot analysis of whole cell extracts of human cerebral cortex from two healthy individuals (#85, #93) (lanes 1 and 2), SNB19 and SF188 human glioma cell lines (lanes 3 and 4) and neuroblastoma cell line (IMR32) (lane 5) with anti-Parc antibody (upper) and anti-actin antibody (lower). (C) Subcellular localization of p53 in neuroblastoma cells (SK-N-AS) transfected with Parc-RNAi or control oligonucleotide. The transfected cells were immunostained with anti-p53 antibody (1801) (visualized by fluorescence) and counterstained with DAPI to visualize the nuclei. (D) RNAi-mediated reduction of Parc in SK-N-AS and IMR32 neuroblastoma cell lines. Whole cell extracts from Parc-RNAi or control oligonucleotide transfected cells, were immunoblotted with anti-Parc, anti-p53 (DO-1), anti-p21 (C19) and anti-actin (AC-15) antibodies. (E) Quantification of subcellular p53 localization (cytoplasmic and nuclear) in SK-N-AS and IMR32 neuroblastoma cells transfected with Parc-RNAi or control oligonucleotide. The data represent the average of three experiments with standard deviations indicated.

FIGS. 8A and 8B The combination of Parc reduction and genotoxic stress strongly activates p53-mediated apoptosis in neuroblastoma cells. (A) Subcellular localization of p53 in neuroblastoma cells (SK-N-AS) transfected with Parc-RNAi or control-RNAi and treated with 0.25 μM etoposide. (B) Enhancement of the DNA damage-induced apoptotic response in neuroblastoma cells by RNAi-mediated Parc reduction. Parc-RNAi or control oligonucleotide transfected SK-N-AS cells were treated with 0.25 μM etoposide and analysed for apoptotic cells (sub-G1) according to DNA content (PI staining).

FIGS. 9A-9E Western blot analysis of the Parc-p53 interaction in various types of cells. (A) Specificity of anti-Parc antibody and co-migration of different forms of Parc. Western Blot analysis of U2OS whole cell extract (lane 1), immunoprecipitates with anti-Parc antibody from whole cell extract (lane 2), immunoprecipitates with pre-immune serum (lane 3) and Flag-Parc immunoprecipitated on M2 beads from Flag-Parc transfected U2OS cells (lane 4) with anti-Parc antibody. (B) Co-migration of transfected Parc and endogenous Parc from p53(175)-associated complexes. Western Blot analysis of M2 immunoprecipitates from Flag-Parc transfected H1299 cells (lane 1), control immunoprecipitates from parental H1299 cells (lane 2), M2 immunoprecipitates from cytoplasmic (lane 3) and nuclear (lane 4) extracts of Flag-p53(175)/H1299 stable cell line. (C) The interaction between Parc and p53 in H460 cells. Western blot analysis of indicated whole cell extract (WCE) (lane 1) or immunoprecipitates with anti-Parc antibody (lane 3) or control immunoprecipitates with preimmune serum (lane 2) from the same extract, with anti-p53 monoclonal antibody DO-1 (lower) or anti-Parc antibody (upper). (D) The interaction between p53 and Parc in H460 cells. Western blot analysis of indicated whole cell extract (WCE) (lane 1) or immunoprecipitates with anti-p53 monoclonal antibody DO-1 (lane 3) or control immunoprecipitates with anti-Ras antibody (lane 2) from the same extract, with anti-Parc antibody. (E) Western blot analysis of Parc and p53 expression levels in breast cancer cell lines. Western blot analysis of whole cell extracts of breast cancer cell lines (lanes 1 through 7), with anti-Parc antibody (upper) or anti-p53 monoclonal antibody DO-1 (middle) or actin antibody (AC-15, bottom). The p53 gene is deleted in one of breast cancer cell lines (MDAMB-453, lane 6). Both MDAMB-468 and MDAMB-435 contain mutated forms of p53 protein, while the MCF-7 cell line expresses wild-type p53.

FIGS. 10A and 10B Parc over-expression abrogates stress-induced nuclear localization of endogenous p53 in U2OS cells. (A) Subcellular localization of p53 in parental U2OS and in Parc-overexpressing (Flag-Parc/U2OS) cell lines after treatment with etoposide (1 μM) for 6 hours. The etoposide-treated cells were immunostained with anti-p53 antibody (1801) (visualized by fluorescence) and counterstained with DAPI to visualize the nuclei. (B) Quantification of subcellular p53 localization in parental U2OS and in Parc-overexpressing (Flag-Parc/U2OS) cell lines after the cells were immunostained with the anti-p53 antibody (1801). More than 90% of U2OS cells displayed nuclear p53 staining after etoposide treatment. Parc-overexpressing (Flag-Parc/U2OS) cells, however, showed less than 30% of nuclear p53 staining after etoposide treatment.

FIGS. 11A-11C RNAi ablation of endogenous Parc induces nuclear localization of a p53 mutant (p53(176)) in SF210 glioma cell line. (A) RNAi ablation of endogenous Parc expression in SF210 cells. Whole cell extracts from Parc-RNAi (lane 2) or control-RNAi (lane 1) treated cells (SF210), were immunoblotted with anti-Parc, anti-p53 (DO-1), and anti-actin (AC-15) antibodies. (B) Quantification of subcellular p53 localization in SF210 cells transfected with Parc-RNAi or control oligonucleotide after the cells were immunostained with the anti-p53 antibody (1801). (C) Subcellular localization of p53 in SF210 cells transfected with Parc-RNAi or control oligonucleotides. The transfected cells were immunostained with anti-p53 antibody (1801) (visualized by fluorescence) and counterstained with DAPI to visualize the nuclei.

FIGS. 12A and 12B Subcellular localization of p53 and Parc in unstressed and etoposide-treated U2OS cells. (A) Subcellular localization of p53 in unstressed U2OS cell line (upper panels) and after treatment with etoposide (1 μM) for 6 hours (bottom panels). The U2OS cells were immunostained with anti-p53 antibody (1801) (visualized by fluorescence) and counterstained with DAPI to visualize the nuclei. (B) Subcellular localization of Parc in unstressed U2OS cell line (upper panels) and after treatment with etoposide (1 μM) for 6 hours (bottom panels). This U2OS cell line overexpresses Flag-Parc. The U2OS cells were immunostained with anti-Parc rabbit polyclonal antibody (visualized by fluorescence) and counterstained with DAPI to visualize the nuclei.

FIGS. 13A-13C These Figures show the cDNA sequence encoding human Parc (GenBank Accession No. AY145132).

FIG. 14 This Figure shows the amino acid sequence of human Parc (GenBank Accession No. AY145132).

### DETAILED DESCRIPTION OF THE INVENTION

Definitions

“Administering” an agent can be effected or performed using any of the various methods and delivery systems known to those skilled in the art. The administering can be performed, for example, intravenously, orally, nasally, via implant, transmucosally, transdermally, intramuscularly, and subcutaneously. Administering can also include the administration, through any means, of a gene therapy vehicle such as a retrovirus (e.g. adenovirus), whose uses for this purpose are well known.

The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“Agents” include, but are not limited to, small molecules, proteins, nucleic acids, carbohydrates, lipids and any other molecules or compounds.

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Anti-sense nucleic acid” shall mean any nucleic acid which, when introduced into a cell, specifically hybridizes to at least a portion of an mRNA in the cell encoding a protein (“target protein”) whose expression is to be inhibited, and thereby inhibits the target protein's expression.

“Catalytic nucleic acid” shall mean a nucleic acid that specifically recognizes a distinct substrate and catalyzes the chemical modification of this substrate.

“DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

“Expression vector” shall mean a nucleic acid encoding a nucleic acid of interest and/or a protein of interest, which nucleic acid, when placed in a cell, permits the expression of the nucleic acid or protein of interest. Expression vectors are well known in the art.

“Parc” means “p53-associated Parkin-like cytoplasmic protein.” As used herein, “Parc” and “p270/Parc” are synonymous.

“Parc expression” means the transcription and/or translation of the Parc gene, and/or the localization of Parc to a site within a cell subsequent to its translation.

“Inhibiting” the expression of Parc means lessening the degree to which the Parc is expressed (e.g., preventing such expression entirely).

“Nucleic acid” shall mean any nucleic acid, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Recombinant protein” shall mean a non-naturally occurring protein produced using recombinant DNA technology.

“Ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

“Specifically inhibit” the expression of a protein shall mean to inhibit that protein's expression (a) more than the expression of any other protein, or (b) more than the expression of all but 10 or fewer other proteins.

“Subject” shall mean any animal, such as a primate, mouse, rat, guinea pig or rabbit. In the preferred embodiment, the subject is a human.

“Suitable conditions” shall have a meaning dependent on the context in which this term is used. When used in connection with contacting an agent to a cell, this term shall mean conditions that permit an agent capable of doing so to enter a cell and perform its intended function. In one embodiment, the term “suitable conditions” as used herein means physiological conditions.

“Therapeutically effective amount” means an amount sufficient to treat a subject afflicted with a disorder or a complication associated with a disorder.

“Treating” a disorder shall mean slowing, stopping or reversing the progression of the disorder and/or a related complication. In the preferred embodiment, “treating” a disorder means reversing the disorder's progression, ideally to the point of eliminating the disorder itself. As used herein in this context, “ameliorating” and “treating” are equivalent.

### EMBODIMENTS OF THE INVENTION

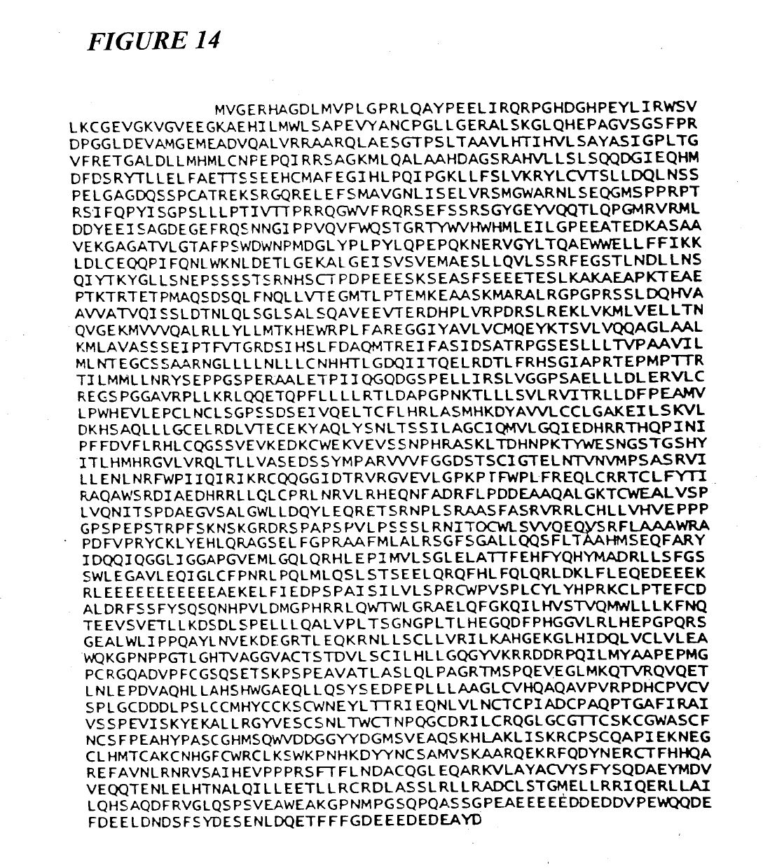

This invention provides an isolated p53-associated Parkin-like cytoplasmic protein (“Parc”). In the preferred embodiment, the Parc is a human protein. For example, the protein can comprise the amino acid sequence set forth in FIG. 14 (Genbank Accession No. AY145132).

This invention also provides a recombinant protein comprising the N-terminal 770 residues of the amino acid sequence set forth in FIG. 14. In one embodiment, this protein consists of the N-terminal 770 residues of the amino acid sequence set forth in FIG. 14.

This invention further provides isolated nucleic acids encoding each of the instant proteins. The instant nucleic acids can be, for example, DNA or RNA. Further provided are an expression vector comprising any of the instant nucleic acids, and a host vector system comprising a cell having therein the instant expression vector. The cell of the host vector system can be, for example, a procaryotic or a eukaryotic cell.

This invention further provides a method for producing a protein comprising culturing the instant host vector system under conditions permitting the expression of the protein encoded by the expression vector therein, and recovering the protein so expressed.

This invention provides a nucleic acid which hybridizes to at least a portion of an RNA encoding Parc. Preferably, the RNA is mRNA, and the Parc is human Parc. In one embodiment, the Parc comprises the amino acid sequence set forth in FIG. 14. In another embodiment, the nucleic acid hybridizes to at least a portion of RNA encoding the N-terminal 770 amino acid residues set forth in FIG. 14.

This invention also provides antibodies, each of which binds to one of the instant proteins. The antibody is preferably detectably labeled, and can be immobilized. Methods for labeling and immobilizing antibodies are well known.

This invention further provides a method for making an antibody which binds to Parc comprising the steps of (a) introducing Parc to a mammal under conditions which permit the generation of antibodies to an antigen; and (b) after a suitable period of time, recovering antibodies generated in the mammal which bind to Parc. Conditions which permit the generation of antibodies to an antigen are well known, and include, for example, the use of adjuvants. Periods of time suitable for permitting the generation of antibodies to a particular antigen in a given species are also well known.

This invention provides a method for determining whether an agent inhibits binding between p53 and Parc comprising

(a) contacting the agent with p53 and Parc under conditions which, in the absence of the agent, permit the formation of a complex between p53 and Parc;

(b) determining the amount of complex formed in step (a) between p53 and Parc; and

(c) comparing the amount of complex determined in step (b) with the amount of complex formed in the absence of the agent, wherein if the amount of complex formed in step (a) is less than the amount formed in the absence of the agent, the agent inhibits binding between p53 and Parc.

In this method, the p53 and Parc are preferably human p53 and human Parc.

This invention also provides a method for determining whether an agent inhibits binding between p53 and the p53-binding portion of Parc comprising

(a) contacting the agent with p53 and the p53-binding portion of Parc under conditions which, in the absence of the agent, permit the formation of a complex between p53 and the p53-binding portion of Parc;

(b) determining the amount of complex formed in step (a) between p53 and the p53-binding portion of Parc; and

(c) comparing the amount of complex determined in step (b) with the amount of complex formed in the absence of the agent, wherein if the amount of complex formed in step (a) is less than the amount formed in the absence of the agent, the agent inhibits binding between p53 and the p53-binding portion of Parc.

In this method, the p53 and p53-binding portion of Parc are preferably human p53 and the p53-binding portion of human Parc. In one embodiment, the p53-binding portion of Parc comprises the N-terminal 770 amino acid residues of Parc set forth in FIG. 14.

In the first two instant assays, any cell-free system permitting Parc and p53 binding can be used. Cell-free systems include, without limitation, the components of a cell, other than an intact cell membrane, required for protein binding. In one example, a cell-free system comprises whole cell extract. In another example, a cell-free system comprises whole cell extract from which cellular organelles have been removed.

This invention further provides a method for determining whether an agent inhibits binding between p53 and Parc in a cell comprising

(a) contacting the agent with the cell under suitable conditions;

(b) determining the amount of complex formed in the cell between Parc and p53; and

(c) comparing the amount of complex determined in step (b) with the amount of complex formed in the absence of the agent, wherein if the amount of complex formed in step (a) is less than the amount formed in the absence of the agent, the agent inhibits binding between p53 and Parc in the cell.

In this method, the cell is preferably a human cell, such as a neuroblastoma cell, a breast cancer cell, a colorectal cancer cell, or a retinoblastoma cell.

This invention further provides a method for determining whether an agent inhibits the expression of Parc in a cell comprising

(a) contacting the agent with the cell under suitable conditions;

(b) determining the amount of Parc expressed in the cell; and

(c) comparing the amount of Parc expression determined in step (b) with the amount of Parc expression in the cell in the absence of the agent, wherein if the amount of Parc expression determined in step (b) is less than the amount of Parc expression in the absence of the agent, the agent inhibits expression of Parc in the cell.

In this method, the cell is preferably a human cell, such as a neuroblastoma cell (e.g., an undifferentiated neuroblastoma cell) a breast cancer cell (e.g., inflammatory breast carcinoma), a colorectal cancer cell (e.g., colorectal carcinoma cell), or a retinoblastoma cell. Also in this method, determining the amount of Parc expression in the cell can be performed using any known method, such as determining the amount of Parc present in the cell or determining the amount of Parc-encoding mRNA in the cell.

This invention also provides a method for decreasing the amount of Parc in a cell comprising introducing into the cell an agent that specifically inhibits the expression of Parc in the cell, thereby decreasing the amount of Parc in the cell.

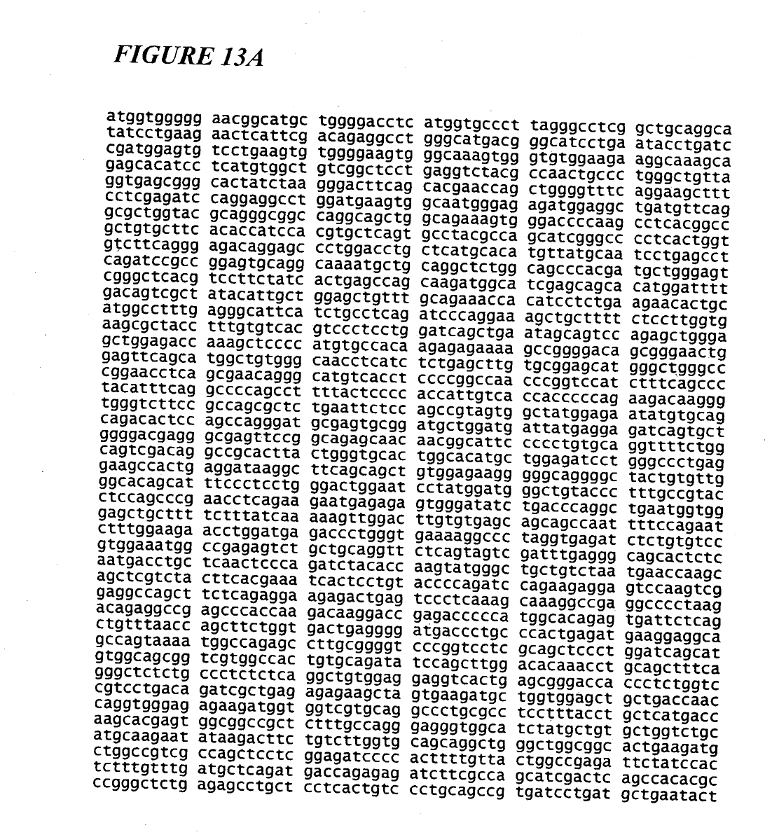

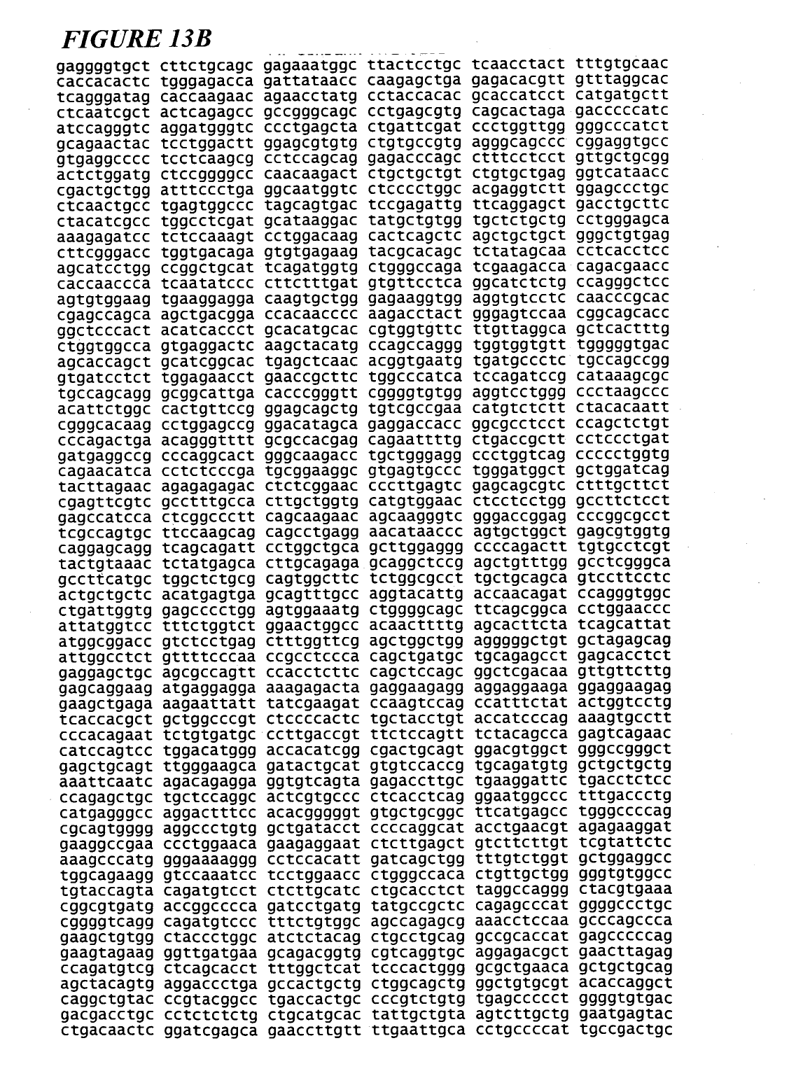

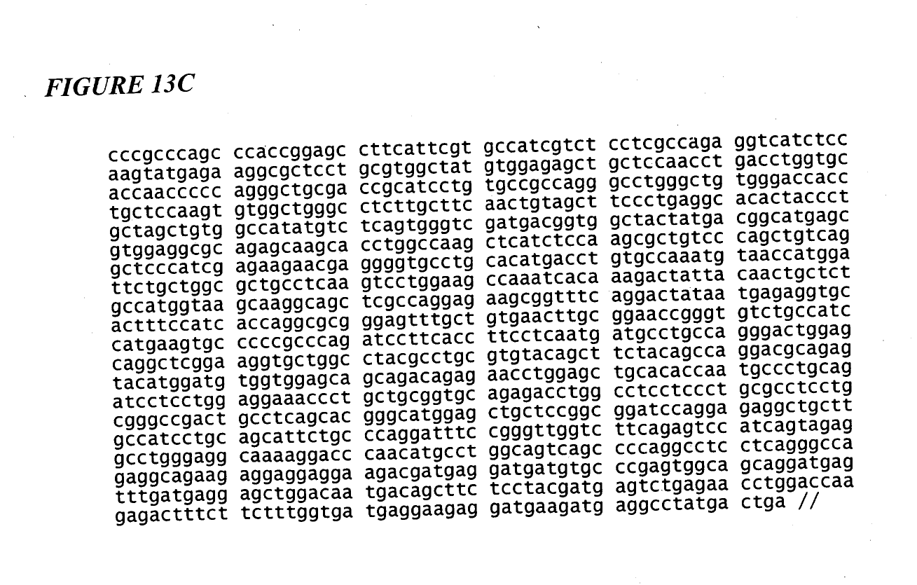

In this method, the cell is preferably a human cell, such as a neuroblastoma cell, a breast cancer cell, a colorectal cancer cell, or a retinoblastoma cell. Moreover, the agent can be, for example, an antisense RNA which hybridizes to Parc-encoding mRNA, a ribozyme which hybridizes to Parc-encoding mRNA, or a DNAzyme which hybridizes to Parc-encoding mRNA. Methods of designing such agents are routine, and can readily be performed based on the mRNA sequence for human Parc as derived from the cDNA sequence set forth in FIGS. 13A-13C (GenBank Accession No. AY145132). Still further, in the preferred embodiment, the cell's p53 is wild-type p53.

This invention further provides a method for treating a subject afflicted with cancer comprising administering to the subject a therapeutically effective amount of an agent that specifically inhibits the expression of Parc in the subject's cells, thereby treating the subject.

In this method, the subject is preferably human, and the subject's cells comprise wild-type p53. The cancer can be any type of cancer including, without limitation, a neuroblastoma, breast cancer, colorectal cancer, or a retinoblastoma. In this method, the agent can be, for example, an antisense RNA, a ribozyme, or a DNAzyme which hybridizes to Parc-encoding mRNA. In one embodiment, the subject is human and the antisense RNA, ribozyme, or DNAzyme hybridizes to the portion of the mRNA which encodes the N-terminal 770 amino acid residues of Parc.

Determining a therapeutically effective amount of agent, be it administered directly or via a gene therapy vector, that specifically inhibits the expression of Parc in a subject's cells can be done based on animal data using routine computational methods.

This invention provides a composition comprising a pharmaceutically acceptable carrier and an agent that specifically inhibits the expression of Parc in a cell. In this composition, the agent can be, for example, an antisense RNA which hybridizes to Parc-encoding mRNA, a ribozyme which hybridizes to Parc-encoding mRNA, or a DNAzyme which hybridizes to Parc-encoding mRNA.

This invention also provides a method for determining whether a cell is cancerous comprising determining the amount of Parc in the cell and comparing the amount so determined to the amount of Parc present in a cell either known to be cancerous or known not to be cancerous, thereby determining whether the cell is cancerous.

In this method, the cell is preferably human, and can be obtained, for example, from a subject suspected of having a neuroblastoma, breast cancer, colorectal cancer, or a retinoblastoma.

This invention further provides a kit for determining the amount of Parc present in a sample comprising a detectably labeled agent which binds to Parc, and instructions for use. In the preferred embodiment, the agent is an antibody, which preferably is immobilized. In addition, the Parc is preferably human Parc.

Finally, this invention provides a kit for determining the amount of Parc-encoding nucleic acid present in a sample comprising a nucleic acid capable of hybridizing to Parc-encoding nucleic acid, and instructions for use. Preferably, in this kit, the nucleic acid is immobilized, and the Parc is human Parc.

This invention is illustrated in the Experimental Details section that follows. This section is set forth to aid in an understanding of the invention but is not intended to, and should not be construed to, limit in any way the invention as set forth in the claims which follow thereafter.

Experimental Details

Synopsis

Nuclear localization of p53 is essential for its tumor suppressor function. Here, we have identified Parc, a Parkin-like ubiquitin ligase, as a cytoplasmic anchor protein in p53-associated protein complexes. Parc directly interacts, and forms a ˜1 MDa complex, with p53 in the cytoplasm of unstressed cells. In the absence of stress, inactivation of Parc induces nuclear localization of endogenous p53 and activates p53-dependent apoptosis. Overexpression of Parc promotes cytoplasmic sequestration of ectopic p53. Furthermore, abnormal cytoplasmic localization of p53 was observed in a number of neuroblastoma cell lines; RNAi-mediated reduction of endogenous Parc significantly sensitizes these neuroblastoma cells in the DNA damage response.

These results reveal that Parc is a critical regulator in controlling p53 subcellular localization and subsequent function.

Methods

Plasmids and Antibodies

To construct Parc expresson constructs, the full-length Parc cDNA or deletion mutants were amplified by PCR from Marathon-Ready HeLa cDNA (Clontech, BD) and subcloned into pcDNA3.1/V5-His-Topo vector (Invitrogen). The Flag sequence was introduced to the N-terminus of Parc by PCR and subcloned into pcDNA3.1/V5-His-Topo vector (Invitrogen). To prepare the Parc antiserum, DNA sequences corresponding to the C-terminal 100 amino acids of Parc (residues 2417-2517) was amplified by PCR (2417-2517) and subcloned into pGEX-2T (Luo et al., 2001). α-Parc antiserum was raised in rabbits against the purified GST-Parc (2417-2517) fusion protein (Covance), and further affinity-purified on the antigen column.

Purification of p53-Interacting Proteins

The epitope-tagging strategy to isolate protein complexes has been described previously (Gu et al., 1999; Luo et al., 2000). To obtain Flag-p53 expressing cell line, we transfected p53-null H1299 cells with pCIN4-Flag-p53(175) and selected for 2 weeks on 1 mg/ml G418 (GIBCO). The p53-expressing colonies were expanded and used for cytoplasmic and nuclear extract preparations essentially as described before (Dignam et al., 1983). For cytoplasmic extract preparation, cells were incubated in buffer A (10 mM HEPES pH 7.9, 10 mM KCl, 0.1 mM EDTA, 0.1 mM EGTA, 1 mM DTT and fresh proteinase inhibitors (Sigma)) for 15 min, NP40 (CALBIOCHEM) was added to 0.5%, and the samples were centrifuged for 5 min at 3000 rpm. Supernatants were filtered with syringe filters (NALGENE). NaCl was added to the supernatants at the final concentration of 200 mM and resulting samples were used as cytoplasmic extracts for M2 immunoprecipitations. Pellets were vortexed for 15 min in buffer C (20 mM HEPES pH 7.9, 400 mM NaCl, 1 mM EDTA, 1 mM EGTA, 1 mM DTT and fresh proteinase inhibitors (Sigma), centrifuged for 10 min at 15000 rpm, filtered with syringe filters (NALGENE). Supernatants were diluted with buffer D (20 mM HEPES pH 7.9, 1 mM EDTA, 1 mM EGTA) to the 200 mM final NaCl concentration and used as nuclear extracts for M2 immunoprecipitations. Affinity chromatography on M2 beads was used to purify Flag-p53(175) complexes. The binding proteins where eluted from the column with Flag peptide and resolved by SDS-PAGE in a 5-15% gradient gel (Novex). Specific bands were cut out from the gel and subjected to mass-spectrometry peptide sequencing.

Ablation of Endogenous Parc by RNAi

H1299, U2OS, IMR32 and SK-N-AS cells were maintained in DMEM medium supplemented with 10% fetal bovine serum. The RNAi-mediated ablation of endogenous Parc was performed essentially decribed as previously (Elbashir et al., 2001). A 21-nucleotide siRNA duplex with 3′ dTdT overhangs corresponding to Parc mRNA (AAGCUUUCCUCGAGAUCCAGG) was synthesized (Dharmacon). The same sequence in the inverted orientation (AAGGACCUAGAGCUCCUUUCG) was used as a non-specific RNAi control. RNAi transfection controls also included Parc sense DNA oligonucleotides and regular plasmids. RNAi transfections were performed using Oligofectamine Reagent (Invitrogen). 24 hours before transfection, about 1 million cells were plated on a 10 cm dish. Cells were transfected using manufacturer's protocol (Invitrogen) for three times with 24-48 hour intervals. After three consecutive transfections, cells were harvested for the western-blot analysis or for flow cytometry analysis, or for immunostaining.

DNA Damage Response and Immunofluorescent Staining

The assay for the DNA damage response was performed essentially as described previously (Luo et al., 2001). Neuroblastoma cells were treated with 0.25 μM etoposide for 8 hours, washed twice with PBS and supplemented with fresh DMEM with 10% FBS. 36 hours after treatment, cells were stained with PI and analyzed by flow cytometry for apoptotic cells (subG1) according to DNA content. For immunofluorescent staining cells were grown on 8-well polylysine slides essentially as described previously (Guo et al., 2000).

For the p53 cytoplasmic sequestration assay, cells were transfected with 30 ng/well CMV-p53 and 150 ng/well CMV-Parc. 24 hours post-transfection cells were fixed with 4% paraformaldehyde for 20 min. on ice, rehydrated for 5 min in serum-free DMEM and permeabilized with 0.2% Triton X100 (Fisher) for 10 min on ice. Cells were incubated in 1% bovine serum albumin (BSA) (Sigma)/Phosphate Buffered Salt solution (PBS) (Cellgro) for 30 min. Primary p53-specific monoclonal (for endogenous p53 immunostaining) (DO-1, 1801, Santa Cruz) or polyclonal (for transfected p53 immunostaining) (FL-393, Santa Cruz) antibodies or anti-Flag monoclonal antibody (for transfected Flag-Parc and Flag-Myc immunostaining) (Santa Cruz) were added in 1% BSA/PBS for 45 min at room temperature. After washing with 1% BSA/PBS, Alexa-488 (or Alexa-568) conjugated anti-mouse (or anti-rabbit for polyclonal primary antibody) (Molecular Probes) antibody was added and incubated for 30 min at room temperature. Finally, cells were counterstained with DAPI to visualize the nuclei essentially as described before (Guo et al., 2000).

Results

Affinity Purification of p53-Containing Protein Complexes From the Cytoplasm of Unstressed Human Cells

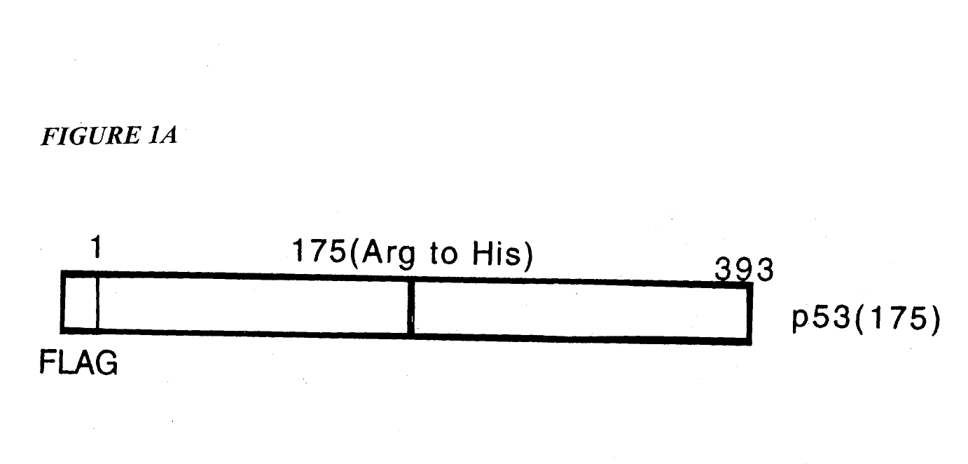

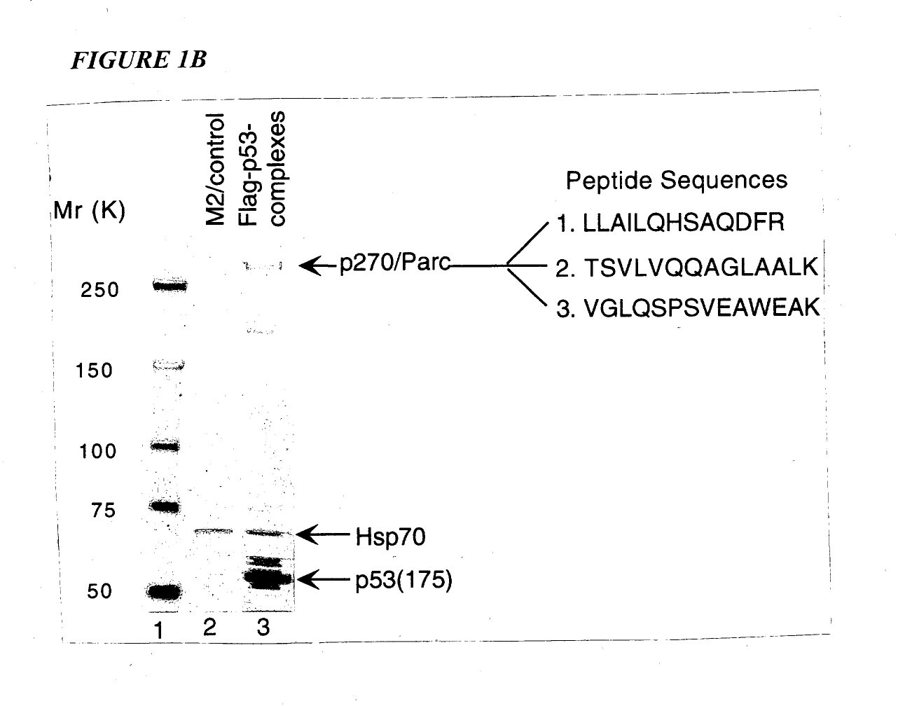

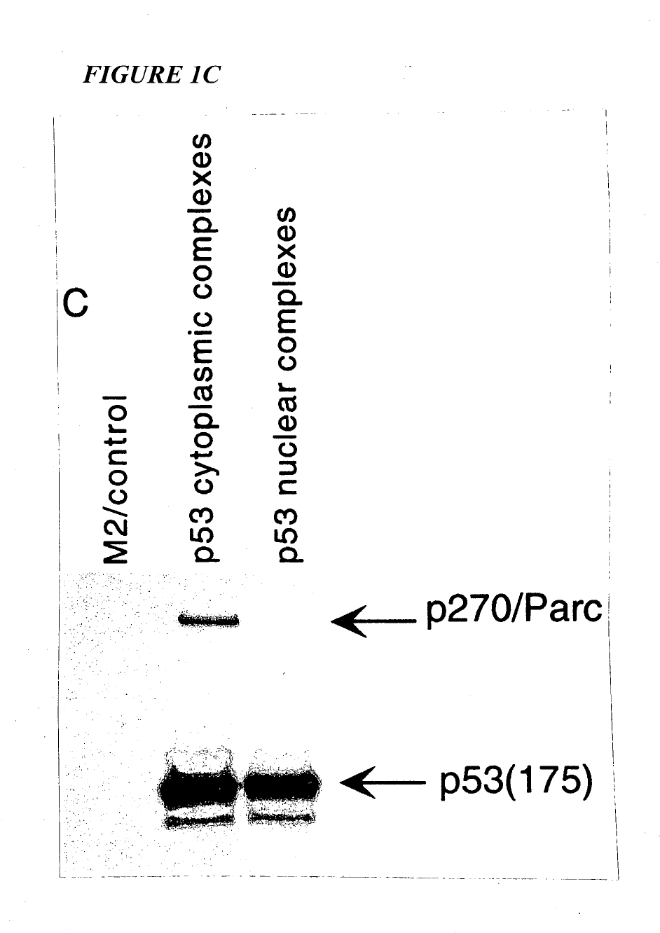

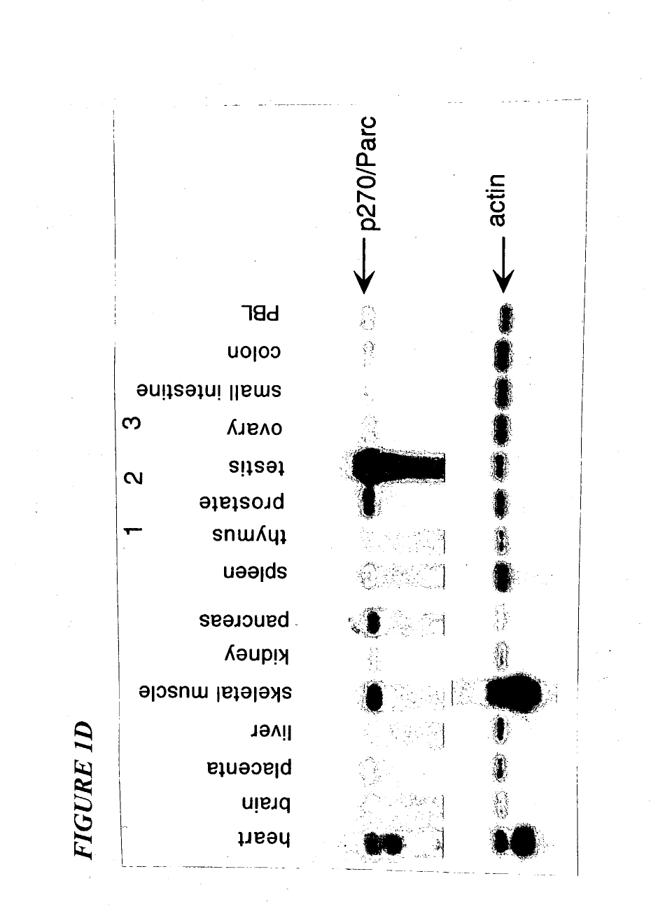

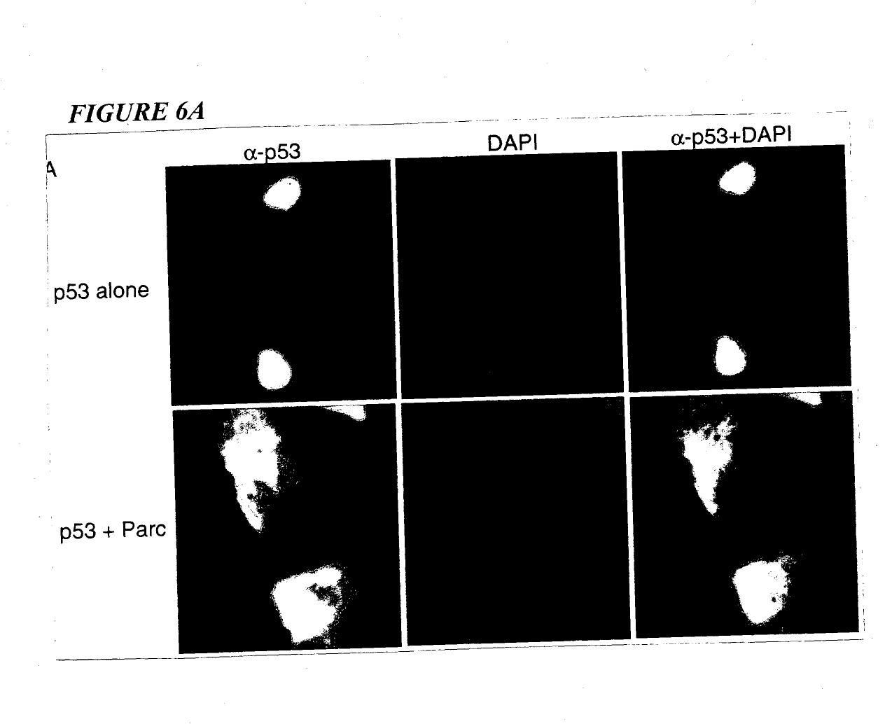

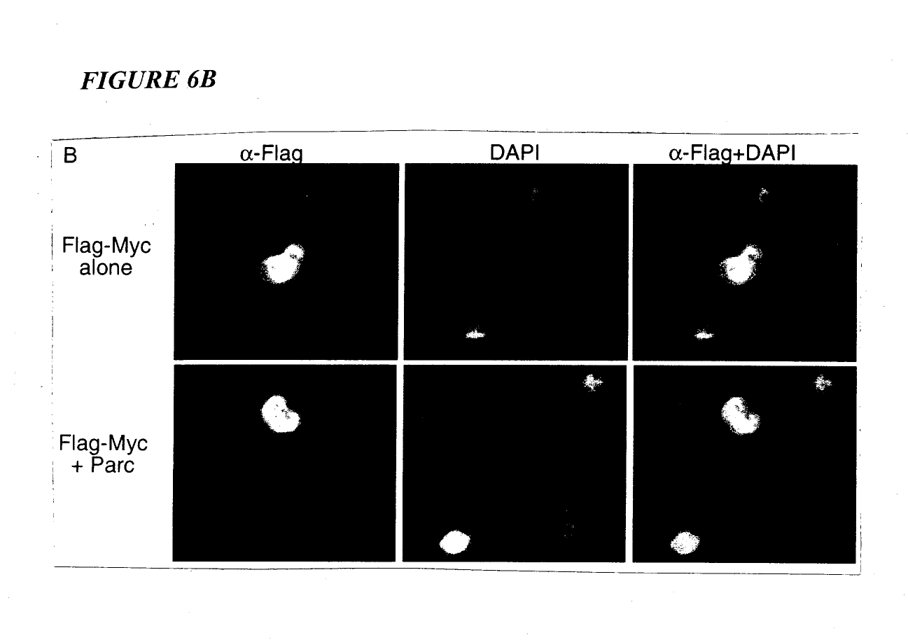

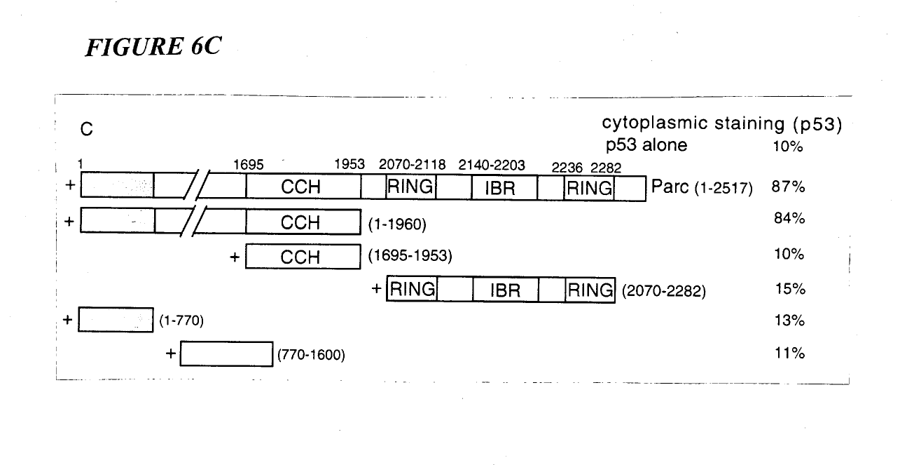

To identify the key cytoplasmic binding partner for p53, we used an epitope-tagging strategy to isolate p53-containing protein complexes from cells. Similar approaches have been used successfully in our lab and others to purify bona fide protein complexes such as the SMCC/TRAP and HDAC1 complexes (Gu et al., 1999; Luo et al., 2000). However, since expression of wild-type p53 strongly induces apoptosis in human cells, it is very difficult to obtain a sufficient quantity of cell extract for further protein purification. Also, a predominantly nuclear pattern of p53 localization is usually obtained when wild-type p53 is ectopically expressed in human cells (Geyer et al., 2000; Boyd et al., 2000, also see FIG. 6). Taken these factors into consideration, we generated a derivative of the human lung carcinoma p53-null H1299 cell line that stably expresses a tumor-derived human p53 mutant (p53(175)) containing a N-terminal FLAG epitope (Flag-p53(175)) (FIG. 1A). Importantly, a significant proportion of the ectopic p53 protein was present in the cytoplasm of this stably-transfected cell line (data not shown, also see FIGS. 1B, 1C).

To isolate protein complexes containing epitope-tagged p53, cytoplasmic extracts from Flag-p53(175)-expressing H1299 cells and from control cells (parental H1299) were subjected to affinity chromatography on M2 (Flag antibody) agarose. As shown in FIG. 1B, a major protein band of ˜270 kDa specifically co-purified with cytoplasmic p53 from Flag-p53(175)-expressing H1299 cells (lane 3) but not from parental H1299 cells (lane 2). Peptide sequencing of this band by mass spectrometry revealed three peptide sequences, all of which matched a single, partial cDNA clone (GenBank accession number Gi4558043, also known as KIAA0708).

Cloning and Initial Characterization of Parc (p53-Associated, Parkin-Like Cytoplasmic Protein)

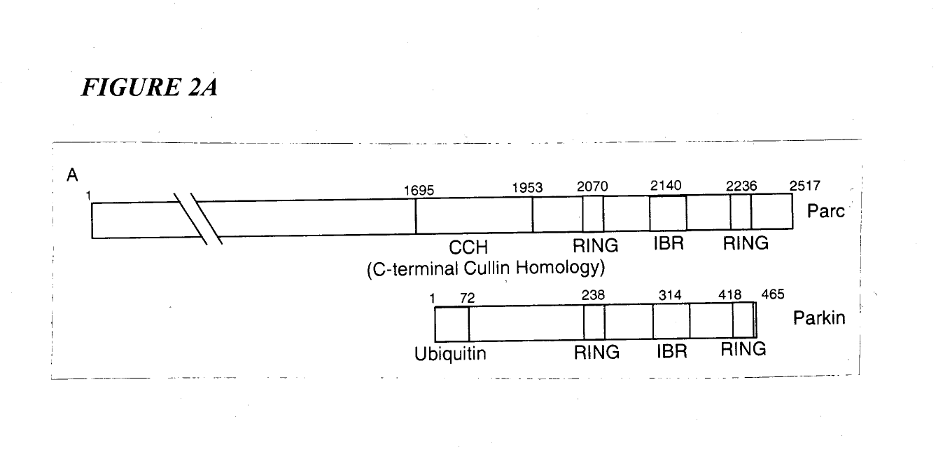

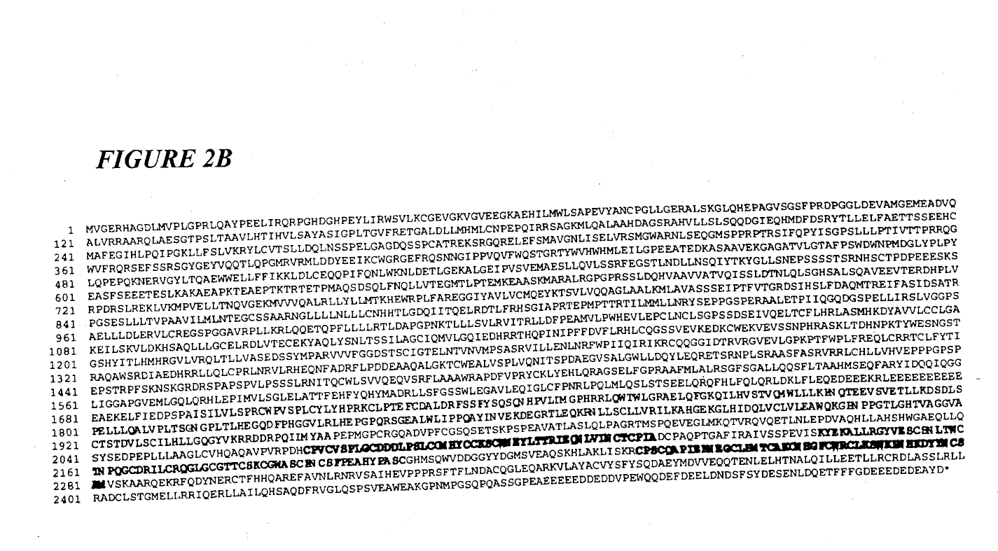

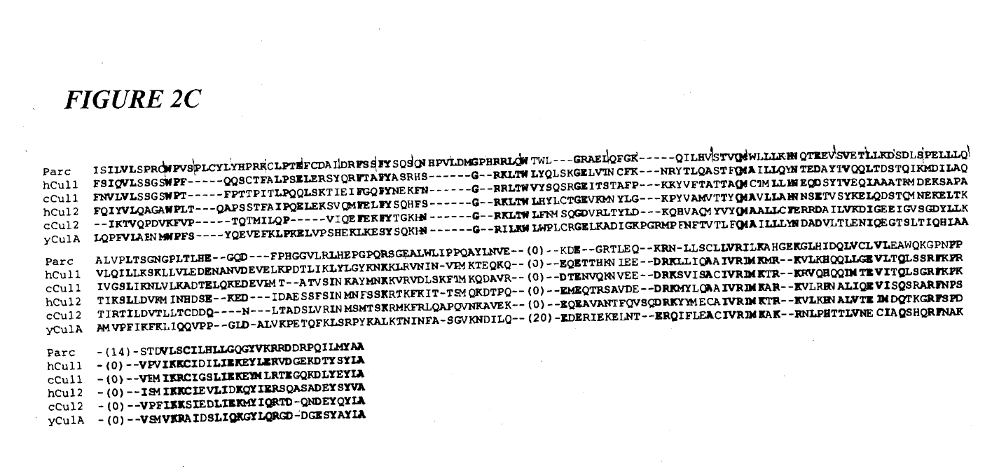

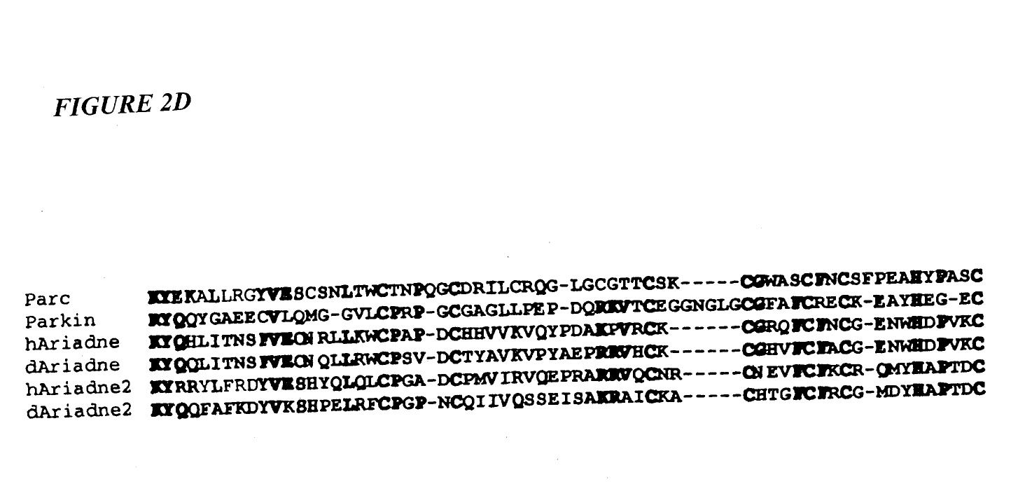

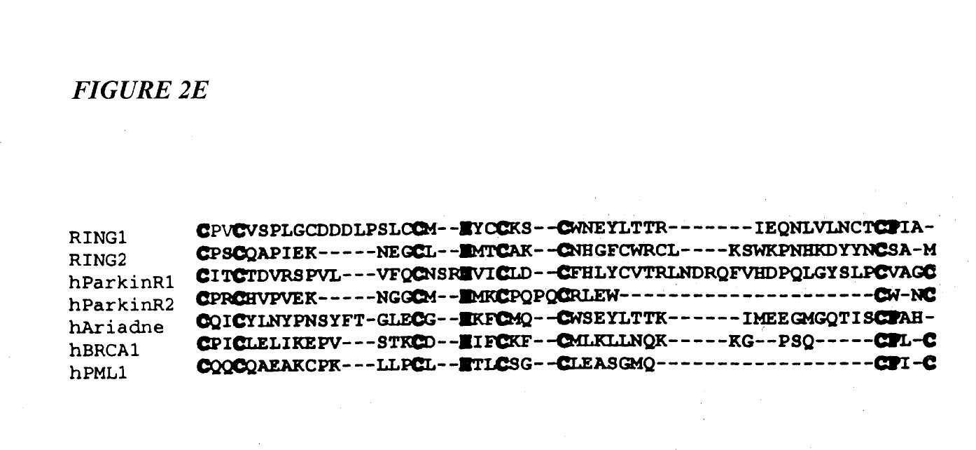

A full-length human p270 cDNA was assembled by exon prediction from genomic sequences, RACE (Rapid amplification of cDNA ends), and homology alignment with the partial sequence of KIAA0708. The full-length cDNA encodes a 2517 amino acid protein (about 800 amino acids longer than the KIAA0708 sequences, FIG. 2B). Northern blot analysis showed that the p270 mRNA is highly expressed in testis but very low in thymus (FIG. 1D). Nevertheless, it is ubiquitously expressed in all different tissues.

Remarkably, the C-terminal sequences of p270 possess a signature motif, the Ring-IBR-Ring domain, that was first identified in the C-terminus of Parkin (FIGS. 2A, 2D, and 2E), the protein implicated in an autosomal recessive form of Parkinson's disease (PD) (Kitada et al., 1998; Shimura et al., 2000). Accordingly, we designated p270 as the p53-associated, Parkin-like Cytoplasmic Protein “Parc.” Biochemical studies have shown that the Ring-IBR-Ring is responsible for the intrinsic ubiquitin ligase activity of Parkin (Shimura et al., 2000). Parkin also contains an ubiquitin-homology domain at the N-terminus that is absent from Parc. Interestingly however, Parc has a unique motif that is highly homologous to the C-terminus of the Cullin proteins (here designated as the C-terminal Cullin Homology CCH domain) (FIG. 2C). The Cullins, including Cul1 and Cu12, are critical components of several ubiquitin ligase complexes, such as the anaphase-promoting complex (APC/C) and the Skp-Cullin-F box complex (SCF) (Pickart, 2001).

Parc Interacts With p53 Both In Vitro and In Vivo

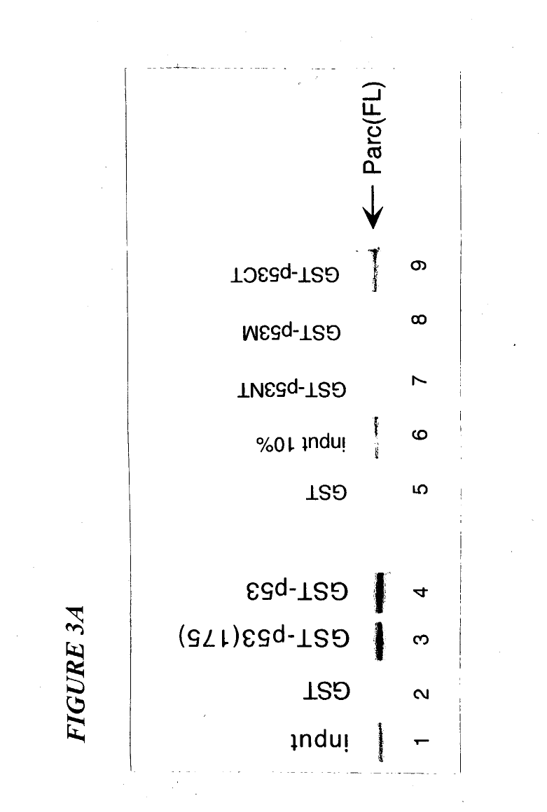

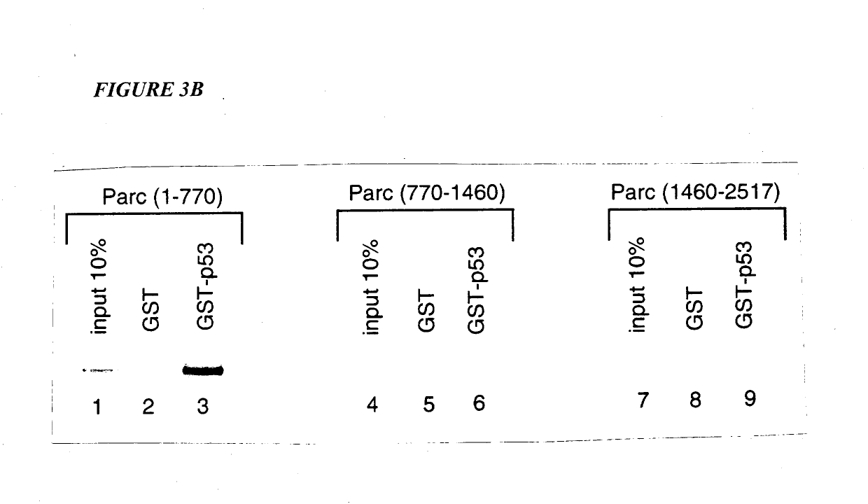

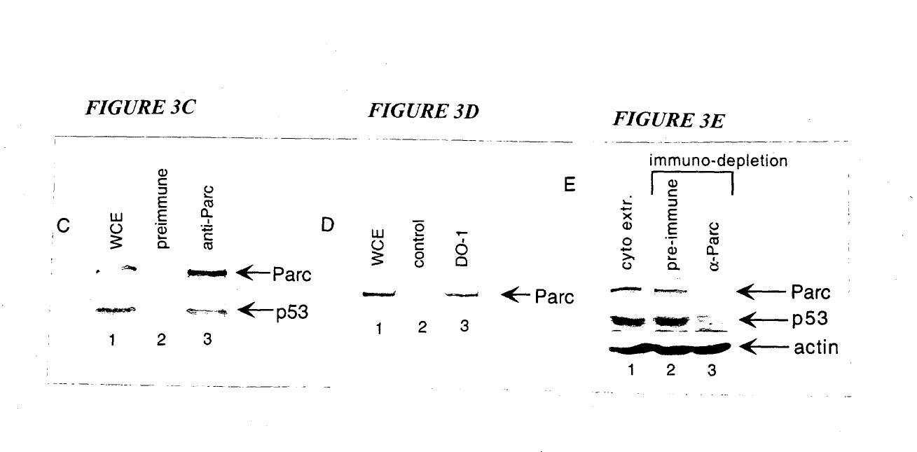

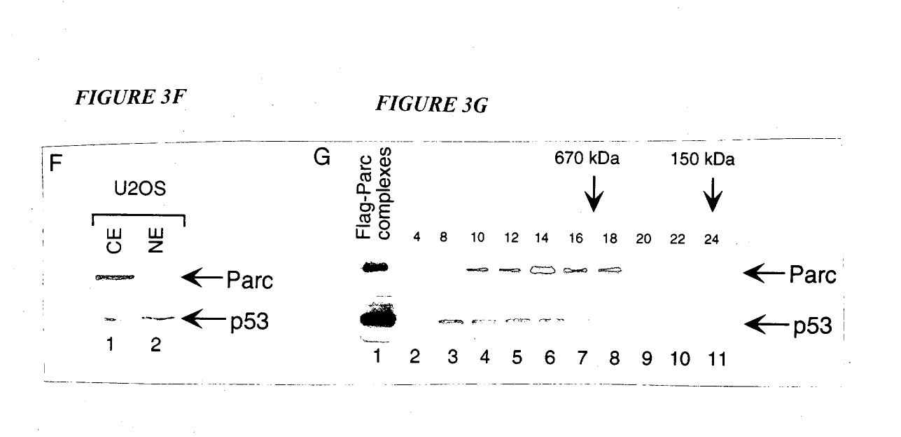

To confirm the physical interaction between p53 and Parc, we first tested whether Parc binds to p53 in vitro. As shown in FIG. 3A, ³⁵S-labeled in vitro-translated Parc bound to immobilized GST-p53 (wild-type) and the p53 mutant form GST-p53(175) (lanes 3, 4), but not to GST alone (lane 2); Parc bound the C-terminus of p53 (GST-p53CT)(lane 9) but showed no binding to the N-terminal domain of p53 (GST-p53NT) (lane 7). A similar strategy was used to map the p53-interacting sequences of Parc. As shown in FIG. 3B, p53 was associated with the N-terminus of Parc. Neither the central region, nor C-terminal region that encompasses the CCH domain and Ring-IBR-Ring motif showed strong binding with p53 (lane 3 vs. lanes 6, 9). Thus, our data indicate that the N-terminus of Parc interacts with the C-terminus of p53.

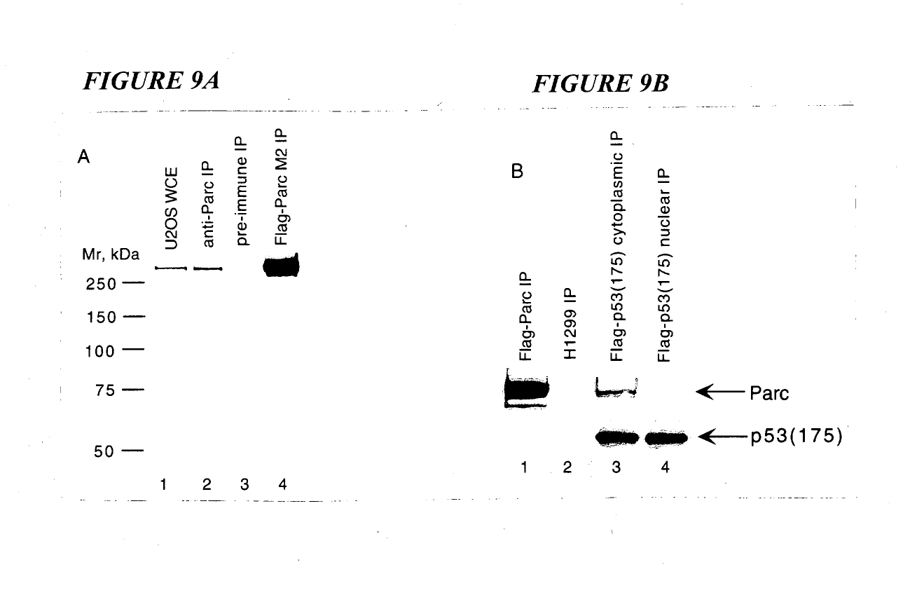

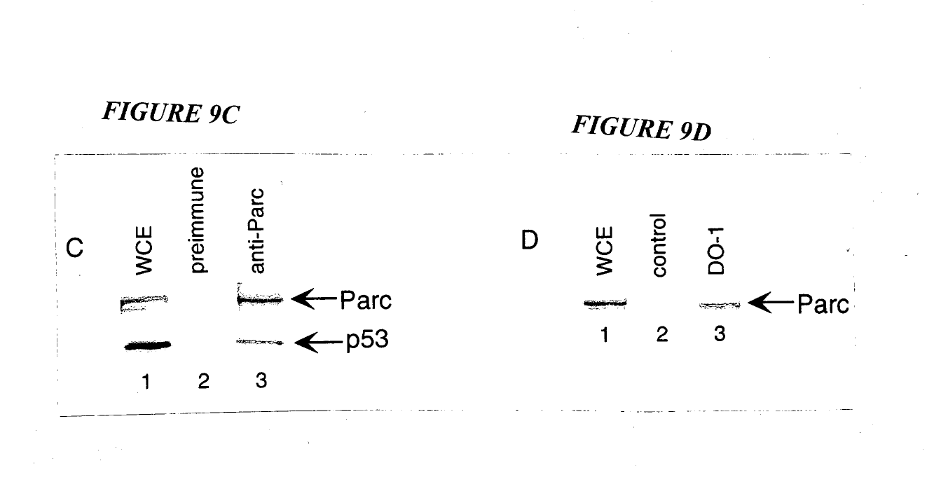

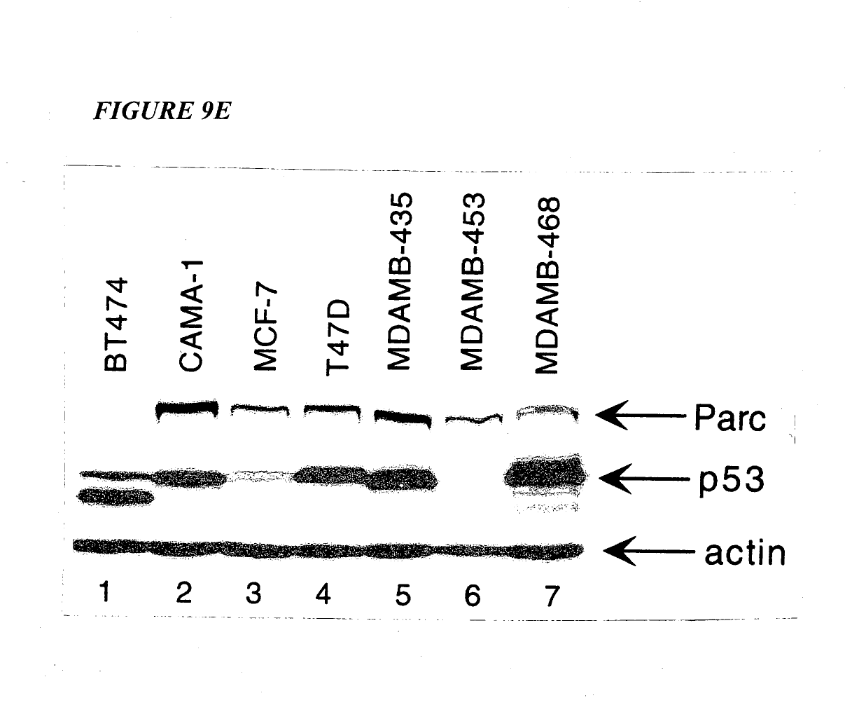

To confirm the interaction between p53 and Parc in vivo, we developed an affinity-purified polyclonal antiserum against the C-terminal 100 residues of Parc (amino acids 2417-2517), a region that shows no apparent homology with any known protein (FIG. 2A). Upon Western blot analysis, this antibody specifically detects Parc proteins in human cell extracts-(lane 1, FIG. 3C). Next, we used this antibody to investigate whether the endogenous Parc and p53 polypeptides interact in vivo. Cell extracts from human U2OS cells, which express wild-type p53, were immunopreciptated with α-Parc or with the corresponding pre-immune serum. As expected, Western analysis revealed that this antibody immunoprecipitated endogenous Parc (lane 3, upper panels, FIG. 3C). More importantly, p53 was clearly detected in the immunoprecipitations obtained with the α-Parc antiserum (lane 3) but not the preimmune serum (lane 2, lower panels, FIG. 3C). Conversely, endogenous Parc was readily immunoprecipated with the p53-specific monoclonal antibody DO-1 (lane 3, FIG. 3D), but not with a control antibody (anti-Ras monoclonal antibody) (lane 2, FIG. 3D). Similar results were also obtained in other cell lines, such as human lung carcinoma H460 cells (see FIGS. 9A-9E). Taken together, these data indicate that p53 and Parc interact both in vitro and in vivo.

Parc Forms a ˜1 MD Complex With p53 in the Cytoplasm

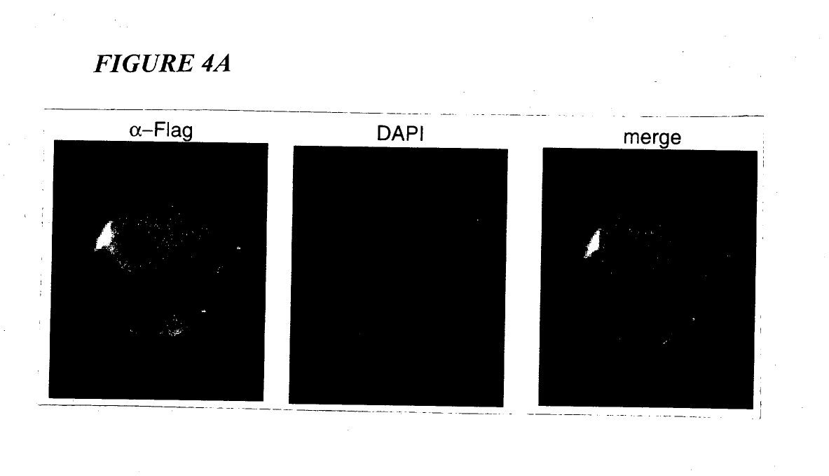

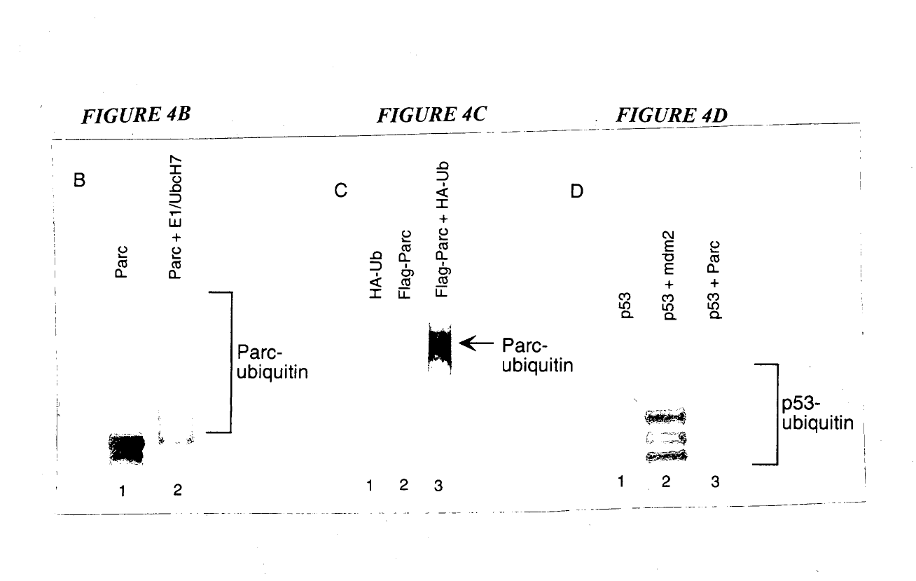

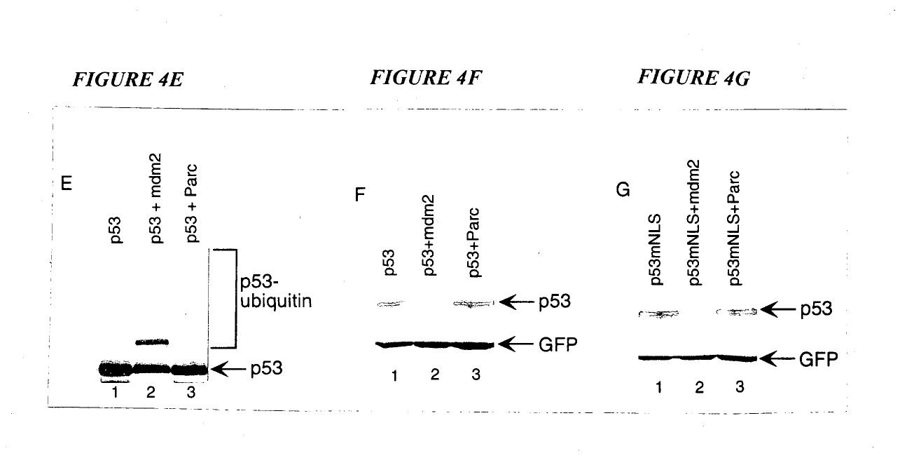

Initial purification and identification of Parc showed that Parc is associated with p53 in cytoplasmic complexes (FIG. 1B). To demonstrate that Parc is a cytoplasmic-specific p53-binding protein, we tested whether p53 is present exclusively in p53-associated cytoplasmic complexes by Western analysis with anti-Parc antibody. As indicated in FIG. 1C, Parc is completely undetectable in p53-associated complexes obtained from nuclear extracts of the Flag-p53(175) stable cell line (upper panels, lane 3 vs. lane 2), although similar levels of p53 were readily immunopreciptitated from both the cytoplasmic and nuclear fractions of these cells (lower panels). Moreover, Western analysis of cellular fractions from native U2OS cells showed that the endogenous Parc protein is present only in cytoplasmic, but not nuclear, extracts (upper panels, FIG. 3F). In accord with these findings, we also observed a predominantly cytoplasmic pattern of immunofluorescent staining in human lung carcinoma H1299 cells transiently transfected with a Flag-tagged Parc polypeptide (FIG. 4A).

Strikingly, through immuno-depletion of the Parc protein from cytoplasmic extracts, we found that more than 70% of cytoplasmic p53 were also co-depleted from the extract, indicating that the majority of cytoplasmic p53 is associated with endogenous Parc (lane 3 vs 1, 2, FIG. 3E). To further evaluate whether Parc can form a cytoplasmic complex with endogenous p53 in unstressed cells, we stably transformed U2OS cells with Flag-tagged Parc. To isolate Parc-containing complexes, the cytoplasmic extract was subjected to affinity chromatography for the Parc-containing complexes. As expected, Western analysis showed that p53 is present in the Parc-containing complexes (lane 1, FIG. 3G). Moreover, when these complexes were subjected to gel-filtration chromatography on Superose 6 (SMART system) as described previously (Gu et al., 1999), p53 and Parc co-eluted in fractions 8-16 with an apparent size of ˜1 MDa (FIG. 3G). Since co-elution of purified protein complexes on gel-filtration chromatography requires a very stable interaction, our data demonstrate that Parc strongly interacts with p53 in the cytoplasm of unstressed human cells.

Parc has an Ubiquitin Ligase Activity but Fails to Directly Induce p53 Degradation

Like Parkin, Parc contains the Ring-IBR-Ring motif (FIG. 2A). Since this motif is required for Parkin-mediated ubiquitination (Shimura et al., 2000), we asked whether Parc also contains an intrinsic ubiquitin ligase activity. As indicated in FIG. 4B, in vitro self-ubiquitination of Parc occurred in the presence of E1, E2 (UBCH7), and ubiquitin (lane 2). Furthermore, highly ubiquitinated species of Parc were readily detected in human cells that co-expressed Flag-tagged Parc and HA-tagged ubiquitin (lane 3, FIG. 4C).

To explore the functional relationship between p53 and Parc, we tested whether Parc directly induces p53 ubiquitination. As indicated in FIG. 4D, a high level of ubiquitinated p53 was found in cells cotransfected with Mdm2 (lane 2) (Li et al., 2002a); however, Parc expression failed to induce significant ubiquitination of p53 (lane 3 vs lane 2). Likewise, using an in vitro assay, we detected strong ubiquitination of p53 by Mdm2, but not by Parc (lane 3 vs. lane 2, FIG. 4E). Moreover, while Mdm2 expression strongly induced p53 degradation, Parc had no effect on the steady-state levels of p53 (FIG. 4F). Notably, we also failed to detect a significant effect of Parc on the cellular levels of p53mNLS (FIG. 4G), a NLS mutant of p53 that, like Parc, resides exclusively in the cytoplasm (data not shown). Thus, Parc is a potential ubiquitin ligase, but fails to directly induce p53 degradation.

RNAi Ablation of Parc Induces Nuclear Localization of p53 and p53-Dependent Apoptosis

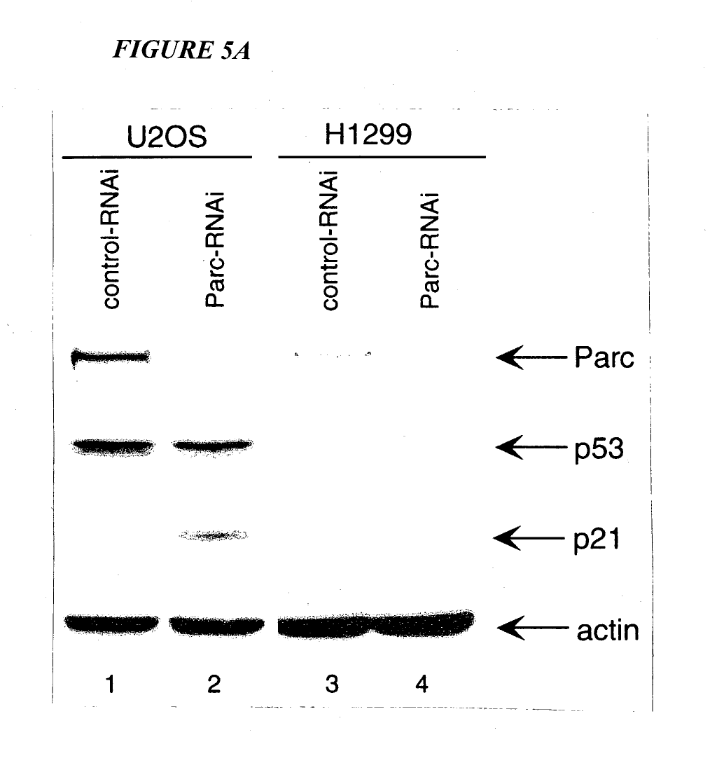

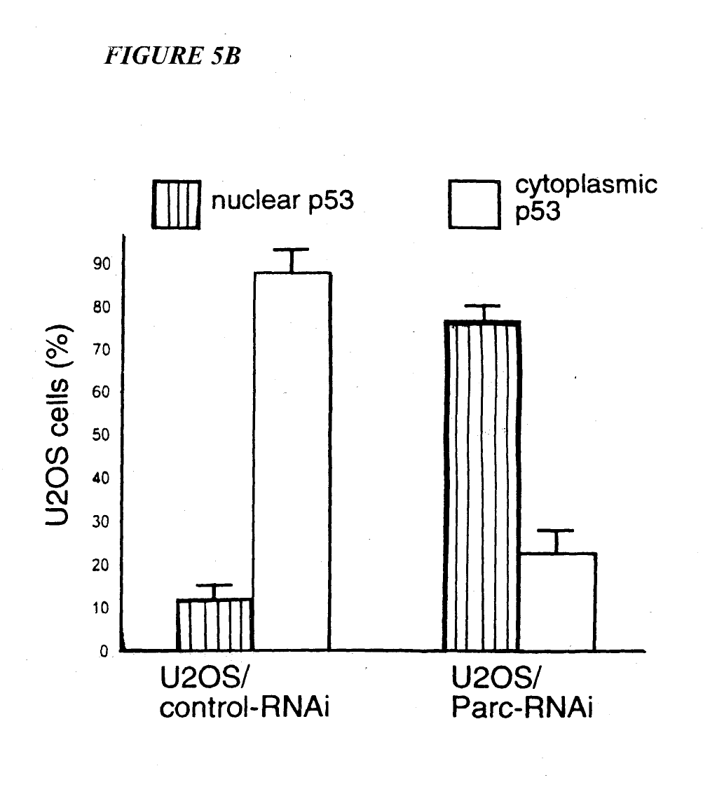

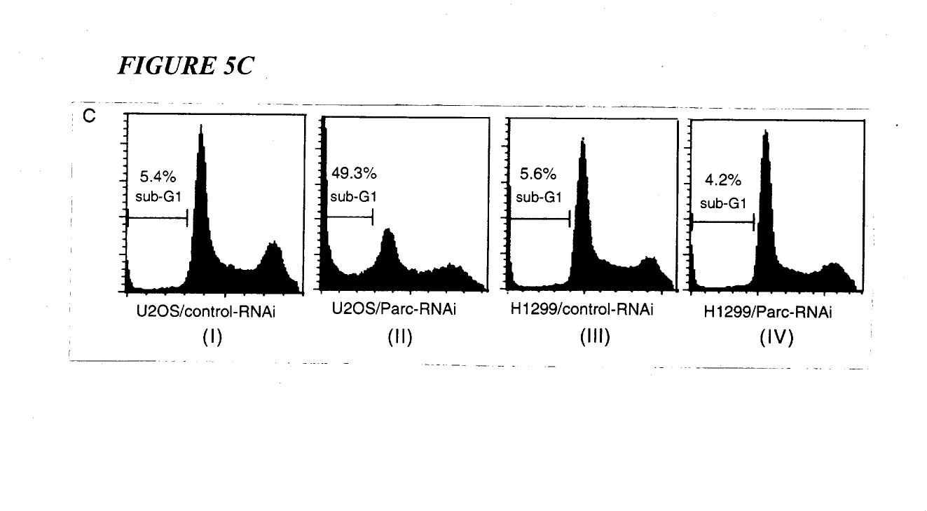

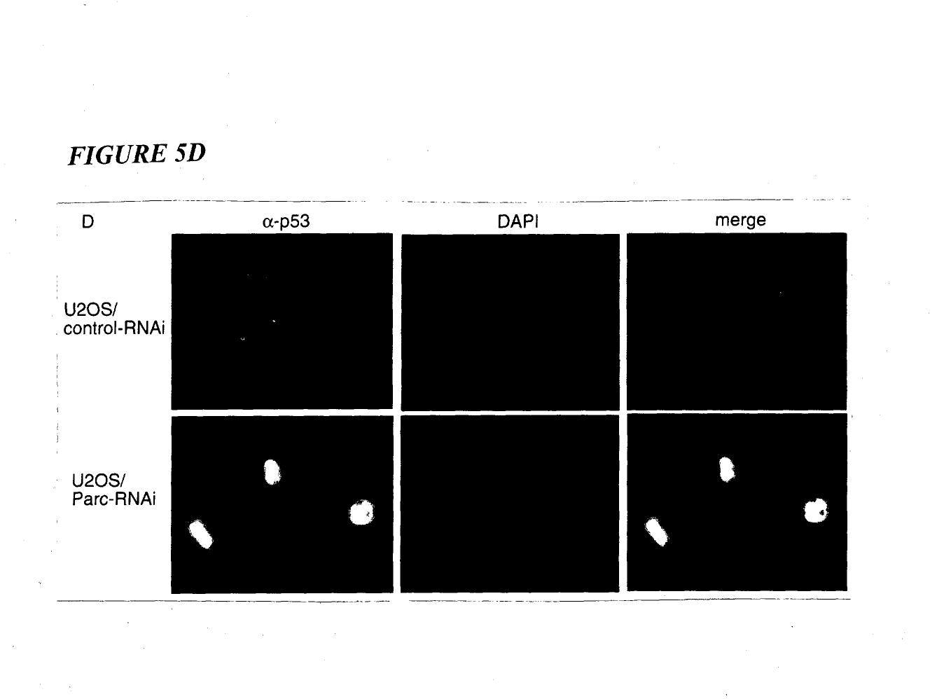

Since we did not observe a significant effect of Parc on p53 ubiquitination, it is likely that Parc may regulate p53-mediated function through other mechanisms. To elucidate the physiological significance of the Parc-p53 interaction, we examined the functional consequences of RNAi ablation of endogenous Parc. For this purpose, U2OS cells, which express both Parc and wild-type p53 proteins (FIG. 3F), were transfected with either a Parc-specific RNA oligonucleotide (Parc-RNAi) or a control oligonucleotide (Control-RNAi). As shown in FIG. 5A, endogenous Parc polypeptides were nearly undetectable after three consecutive oligofectamine-mediated transfections (upper panels, lane 2 vs. lane 1) while the level of control protein (actin) remained the same (lower panels). Strikingly, ablation of Parc expression strongly induced the expression of p21, one of the key transcriptional targets of p53, despite the fact that the steady-state levels of p53 were unchanged (middle panels, Lane 2 vs. Lane 1, FIG. 5A).

Next, we examined the effect of Parc on p53-mediated apoptosis. As shown in FIG. 5C, Parc-RNAi treated cells were susceptible to programmed cell death, with about 49.3% of the cells apoptotic (II) while the control transfected U2OS cells (control-RNAi) showed no significant apoptosis under the same conditions (I). Similar results were also obtained with the TUNEL assay (data not shown). Furthermore, the same experiments were also performed in the human p53-null cell line H1299. As shown in FIG. 5A, although endogenous Parc expression was successfully abrogated by RNAi, neither p21 activation nor significant apoptosis was detected in these cells (lanes 3, 4, FIG. 5A; III and IV, FIG. 5C). These results indicate that ablation of endogenous Parc expression significantly induces p53-mediated transcriptional activation and activates p53-dependent apoposis.

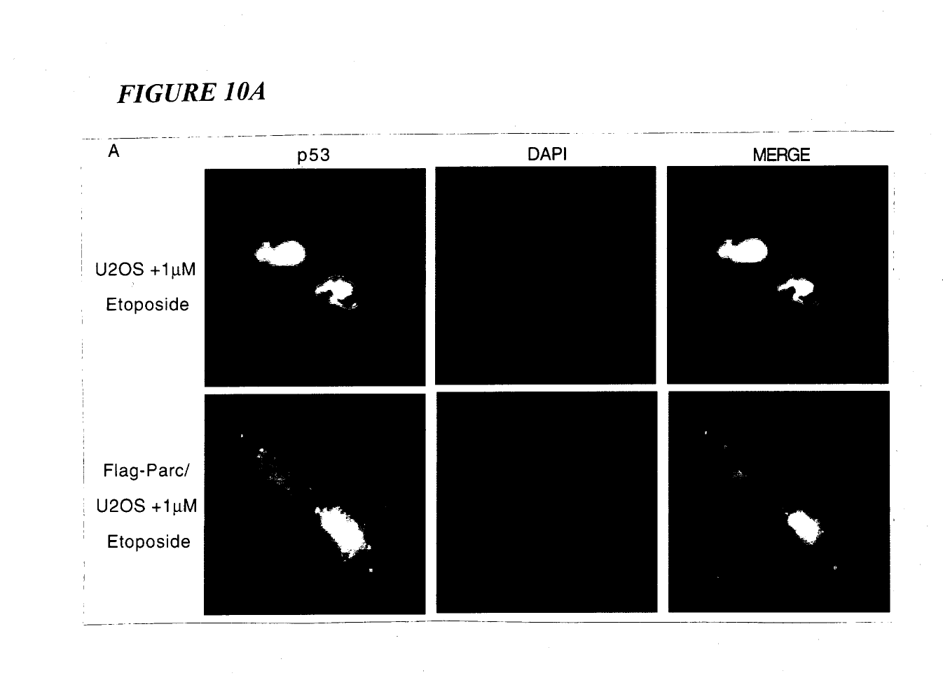

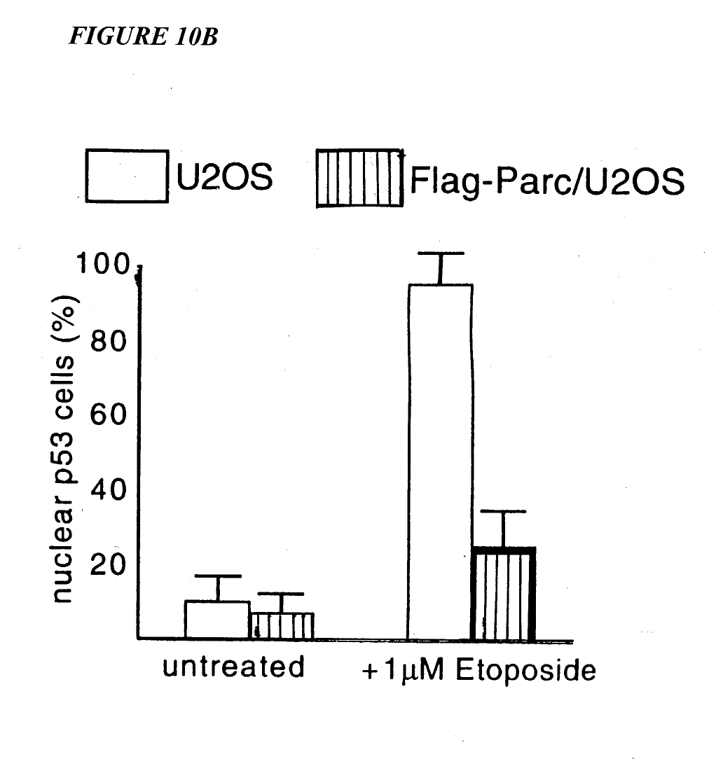

To explore the molecular mechanism by which Parc affects p53 function, we tested whether Parc directly controls subcellular localization of p53. Under normal conditions, p53 is diffusely distributed in the cytoplasm of U2OS cells, as shown by immunofluorescence staining with a p53-specific monoclonal antibody (1801)(upper panels, FIG. 5D) Strikingly however, after RNAi ablation of endogenous Parc, p53 was predominantly relocalized to the nucleus of these cells (lower panels, FIG. 5D); the exclusive nuclear staining of p53 was found in as many as 80% of total Parc-RNAi-treated cells compared with only 12% of control cells (FIG. 5B). Similar results were also obtained in the cells expressing mutated p53 proteins (see FIGS. 10A and 10B). These results indicate that RNAi ablation of Parc expression activates p53-mediated function by regulating its subcellular localization.

Overexpression of Parc Induces Cytoplasmic Sequestration of p53

To elucidate the mechanism by which Parc affects subcellular localization of p53, we investigated whether Parc overexpression directly promotes cytoplasmic retention of p53. As indicated in FIG. 6A, ectopic expression of wild-type p53 yields predominantly nuclear staining (upper panels), with less than 10% of cells showing clear cytoplasmic localization (Geyer et al., 2000; Boyd et al., 2000). However, overexpression of full-length Parc strongly induced cytoplasmic retention of p53, with more than 87% of cells showing cytoplasmic p53 localization (lower panels, FIGS. 6A; 6C). In contrast, Parc had no effect on another nuclear protein, c-Myc (FIG. 6B) indicating that Parc-mediated cytoplasmic sequestration is specific for p53.

To identify the sequences of Parc that are responsible for cytoplasmic retention of p53, the same assay was used to evaluate selected segments of the Parc polypeptide. As summarized in FIG. 6C, the subcellular localization of p53 was not altered by overexpression of individual Parc segments corresponding to either the CCH domain (residues 1695-1953), the Ring-IBR-Ring motif (residues 2070-2282), or two N-terminal regions (residues 1-770 or 770-1600). In contrast, however, a segment encompassing both the N-terminal sequences and the CCH domain (residues 1-1960) readily induced cytoplasmic relocalization of p53. These data also indicate that the Ring-IBR-Ring motif is not required for Parc-mediated sequestration of p53.

Reduction of Endogenous Parc in Neuroblastoma Cells Also Induces p53 Nuclear Localization

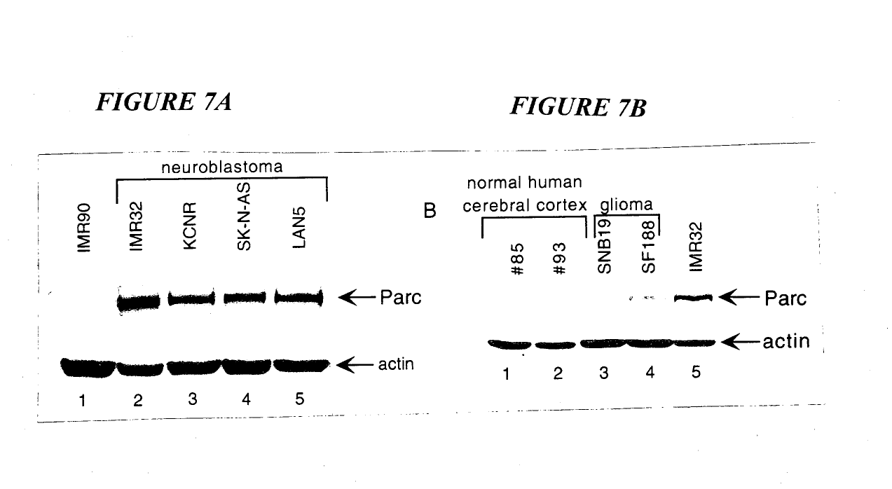

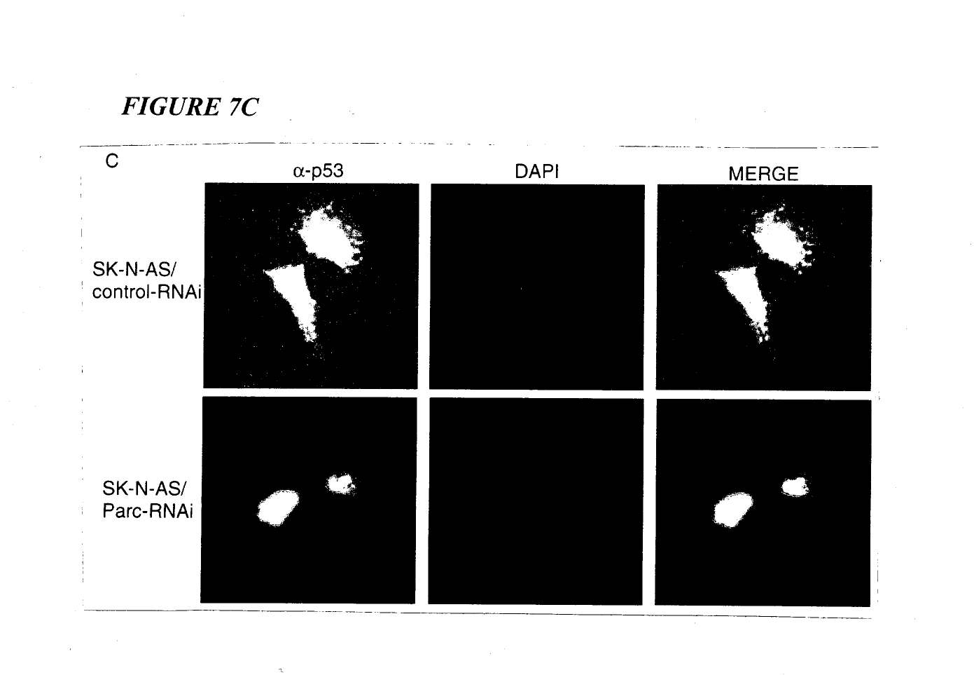

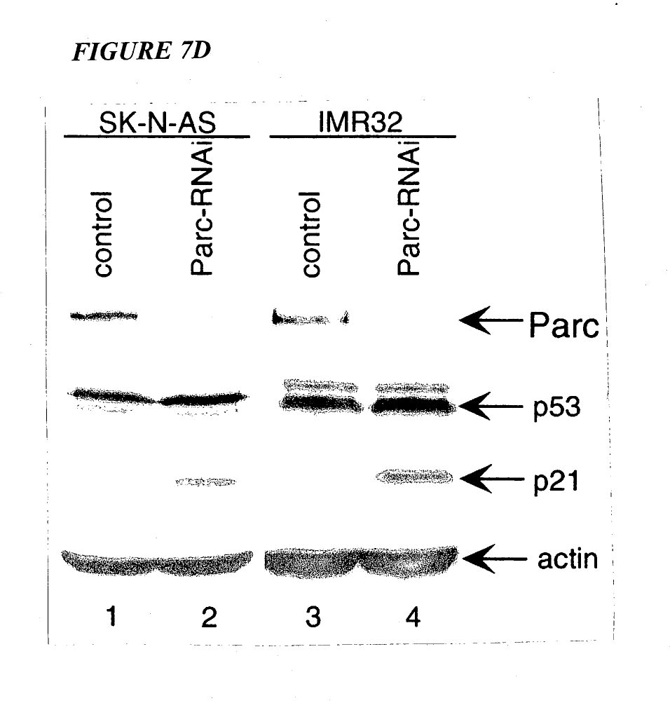

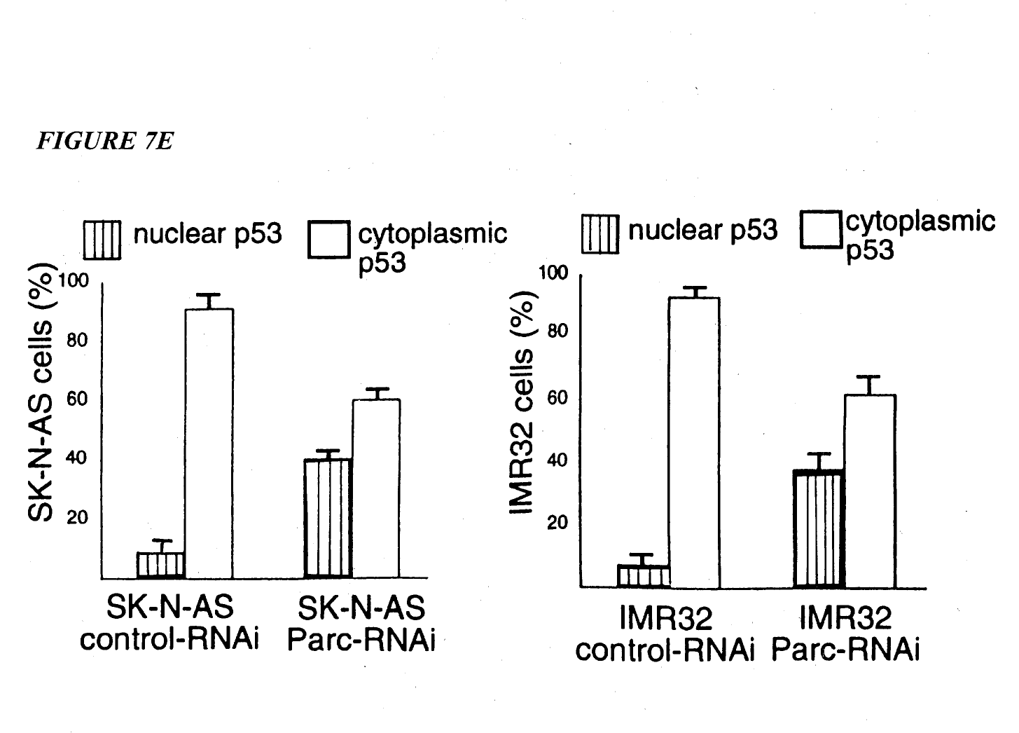

Many neuroblastoma cell lines display predominantly cytoplasmic localization of wild-type p53 (Moll et al., 1996; Sengupta et al., 2000; Stommel et al., 1999; Zaika et al., 1999). The mechanisms by which p53 is sequestered in the cytoplasm of these cells are not completely understood. Interestingly, as indicated in FIGS. 7A, B, the Parc protein is highly expressed in the neuroblastoma cell lines that we tested, including IMR32, KCNR, SK-N-AS and LAN5, compared to either normal human brain tissues or human glioma cell lines.

To explore the role of Parc in this phenomenon, we tested whether inactivation of endogenous Parc expression by RNAi modulates the subcellular localization of p53 in the SK-N-AS and IMR32 neuroblastoma lines. As indicated in FIG. 7D, the protein levels of endogenous Parc in these cells were significantly decreased by Parc-RNAi treatment (upper panels), although the reduction was less than that obtained by the same technique in U2OS cells (FIG. 5A). This presumably reflects the higher steady-state levels of endogenous Parc and lower transfection efficiency of neuroblastoma cells. Nevertheless, RNAi-mediated reduction of endogenous Parc also leads to nuclear localization of p53 in both SK-N-AS and IMR32 cells; the typical punctuated pattern of cytoplasmic p53 staining was converted to a predominantly nuclear staining pattern in about 35% of cells, compared to less than 10% in the control cells (FIGS. 7C, E) Furthermore, expression of p21, a major transcriptional target of p53 was significantly induced despite the fact that the total level of p53 polypeptides was unchanged by Parc-specific RNAi treatment (middle panels, FIG. 7D).

RNAi Mediated Parc Reduction Restores a Strong p53-Dependent Stress Response in Neuroblastoma Cells

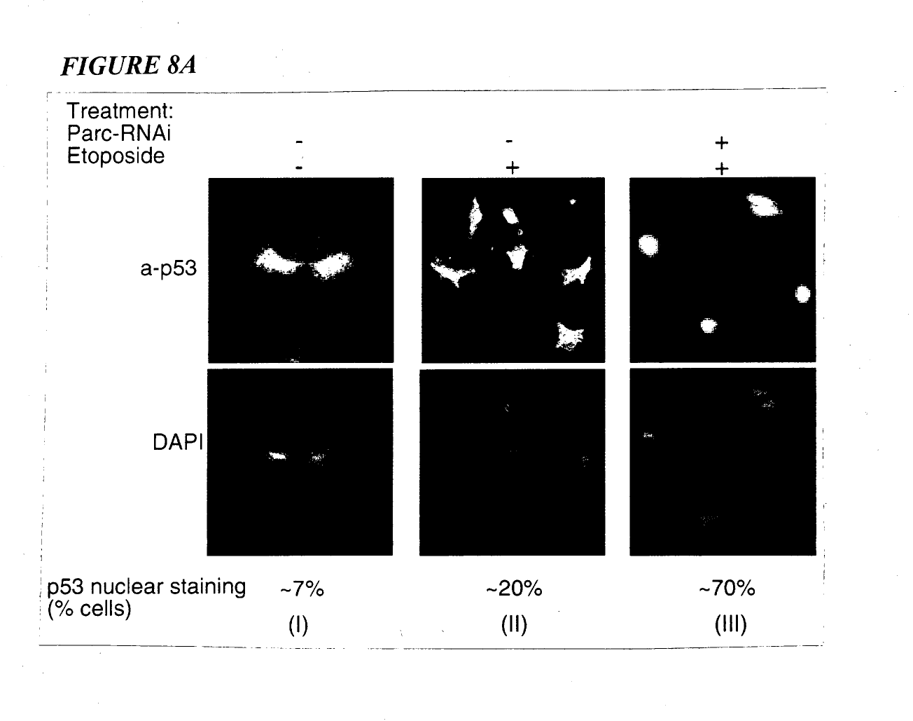

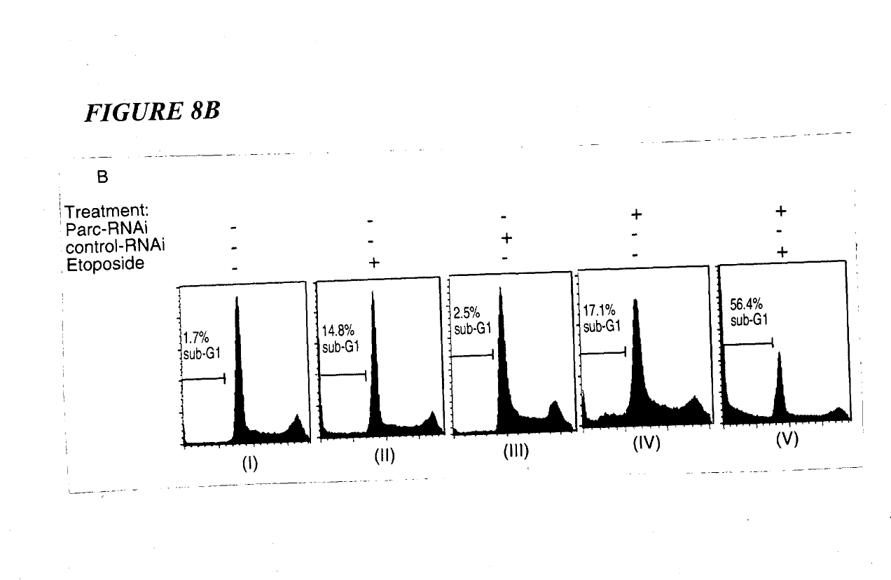

Neuroblastoma cells and other types of tumor cells that show constitutive cytoplasmic localization of p53 often exhibit an impaired response to low levels of DNA damage treatment (Moll et al., 1996). Therefore, we tested whether a normal DNA damage response could be restored in neuroblastoma cells by RNAi-mediated inhibition of Parc function. As indicated in FIG. 8, native SK-N-AS neuroblastoma cells responded poorly to treatment with low levels of the genotoxic drug etoposide; only 20% of cells showed nuclear localization of p53 (II, FIG. 8A) and less than 15% became apoptotic (II, FIG. 8B). However, after treatment with Parc-RNAi, almost 70% of the cells showed nuclear staining of p53 (III, FIG. 8A) and more than half (56.4%) became apoptotic with the same dose of etoposide (V vs. I, FIG. 8B), whereas Parc-RNAi treatment alone only induced apoptosis in 17.1% of cells (IV, FIG. 8B). Thus, the combination of RNAi-mediated Parc reduction and DNA damage restores a strong p53-mediated apoptotic response in neuroblastoma cells.

Discussion

The present data reveal the existence of a key cytoplasmic protein Parc (p53-associated, Parkin-like Cytoplasmic Protein) that is critically involved in the regulation of p53 subcellular localization and subsequent function. As a transcription factor, nuclear localization of p53 is essential for its role in tumor suppression (Vousden, 2002; Jimenze et al., 1999). However, p53 is diffusely distributed in the cytoplasm of normal unstressed cells; many types of tumors cells including neuroblastoma cells have abnormal cytoplasmic localization of p53 and an impaired p53-dependent stress response despite the fact that they express wild-type p53 proteins (Reviewed in Jimenez et al., 1999; Moll et al., 1992, 1995, 1996). We show that the majority of cytoplasmic p53 is tightly associated with endogenous Parc. In the absence of stress, inactivation of endogenous Parc leads p53 activation through inducing p53 nuclear localization. Furthermore, our results also indicate that the levels of Parc proteins are relatively high in neuroblastoma cells. It is conceivable, therefore, that the high levels of Parc in these cells may prevent nuclear translocation of p53, even in the presence of genotoxic stress although other mechanisms may also contribute to this phenomena (Stommel et al., 1999). In consistence with this notion, we have also shown that RNAi-mediated reduction of Parc protein levels can restore a strong p53-dependent stress response in these neuroblastoma cells. As such, the Parc-mediated pathway of p53 regulation may prove to be a potential target for cancer therapy. In particular, agents that down-regulate Parc protein levels or abrogate the Parc-p53 interaction may sensitize tumor cells to p53-dependent apoptosis.

Parc Plays a Critical Role in the Regulation of Subcellular Localization of p53

In unstressed cells, p53 is diffusely distributed, and p53-mediated function appears to be severely inhibited. While no obvious effect on the total p53 protein levels by RNAi-mediated ablation of endogenous Parc, the p53 polypeptides are relocated to the nucleus and p53-mediated functions are also strongly activated in the absence of stress. Conversely, overexpression of Parc induces cytoplasmic sequestration of p53. Therefore, we propose that Parc serves as an anchor protein that tethers p53 in the cytoplasm and thereby regulates p53 subcellular localization.

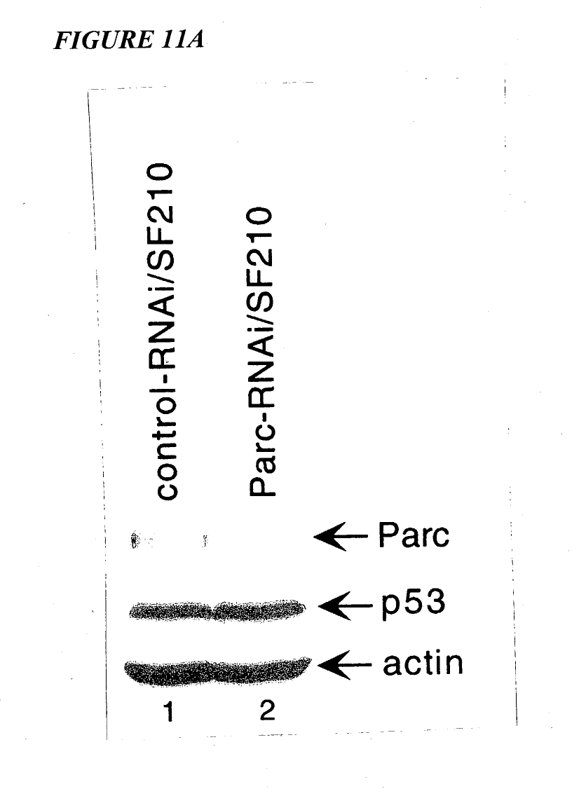

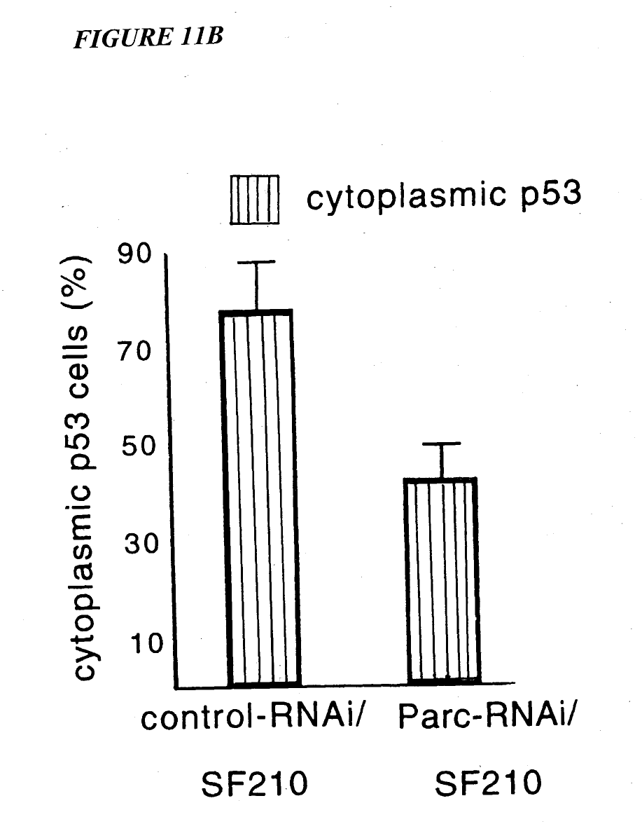

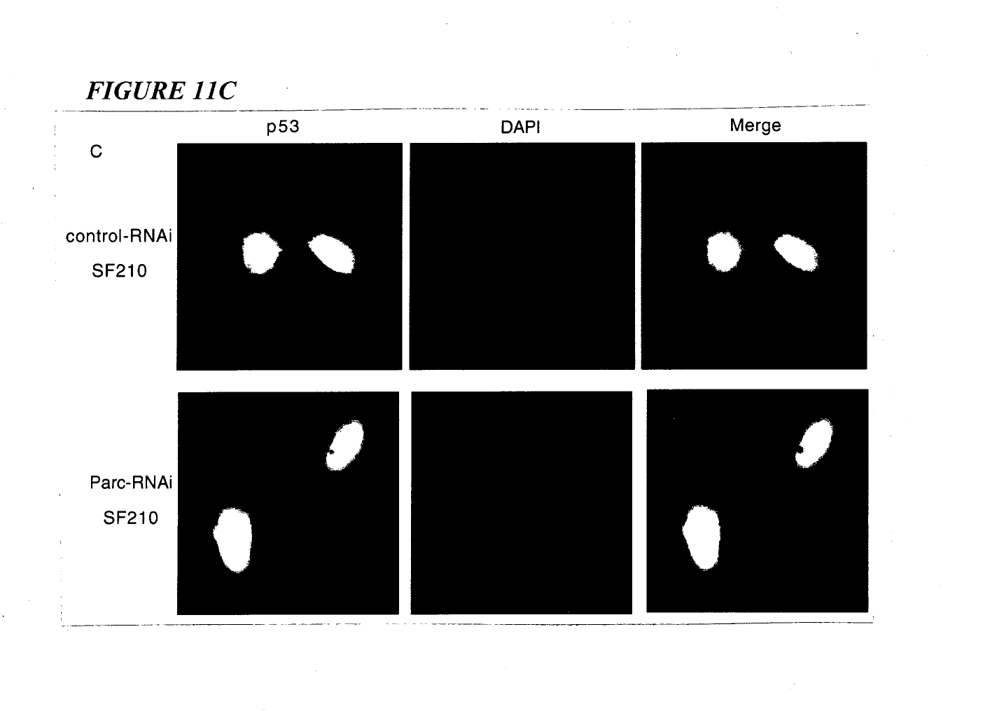

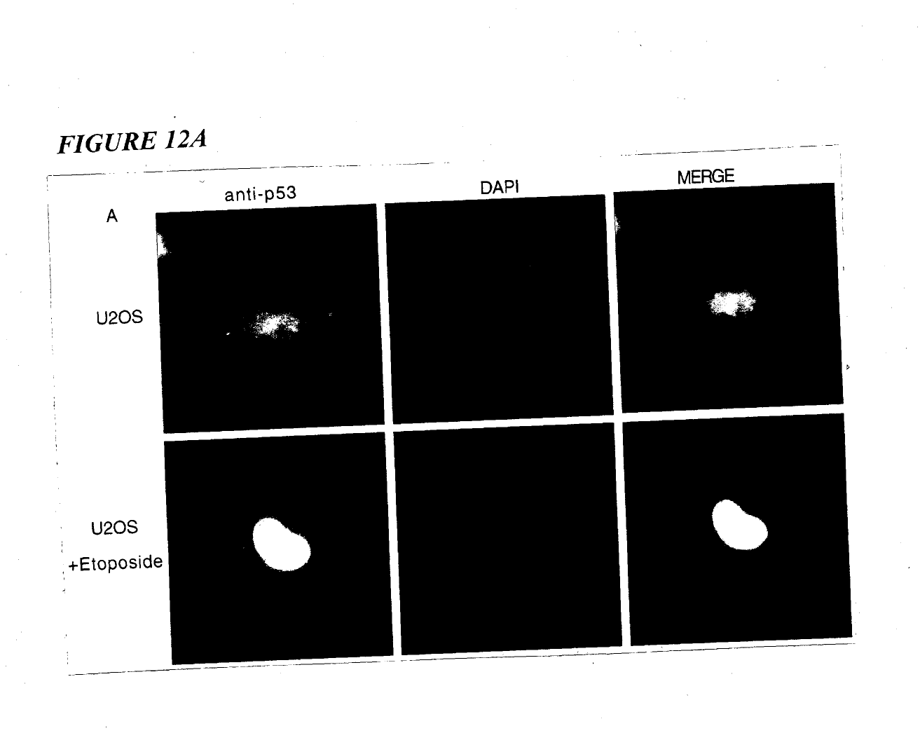

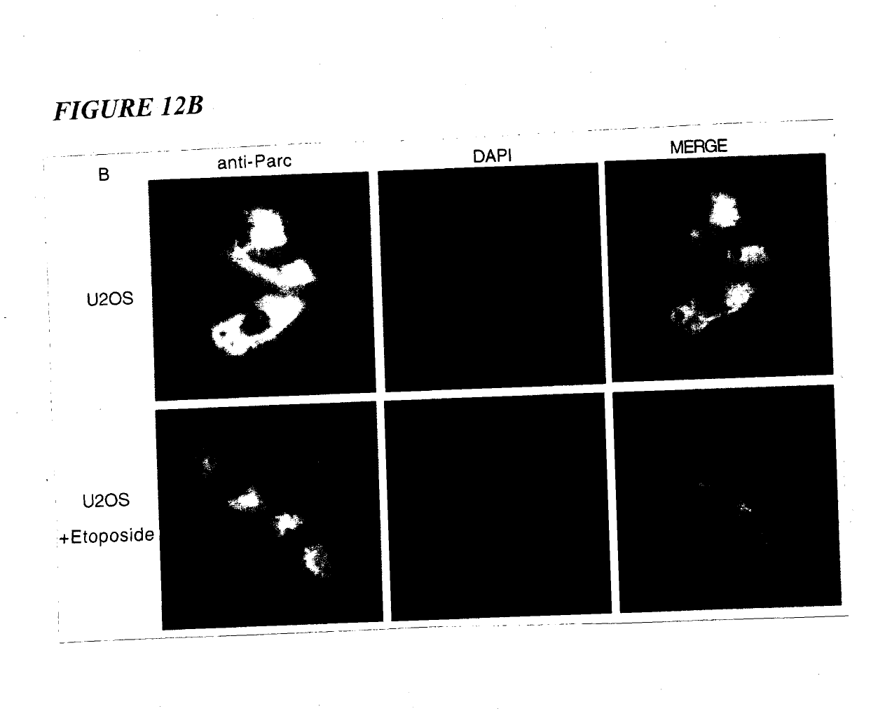

Parc is a constitutive cytoplasmic protein that strongly interacts with the C-terminal domain of p53. Since this domain harbors the three known NLS sequences of p53, it is conceivable that Parc blocks nuclear import of p53 by concealing its C-terminal NLS motifs. Interestingly, overexpression of the p53-binding domain of Parc (residues 1-770) alone is not sufficient to induce cytoplasmic retention of p53 (FIG. 6). Thus, it is likely that additional Parc sequences such as the CCH domain facilitate cytoplasmic sequestration of p53, perhaps by linking the Parc protein to stable cytoplasmic complexes/or structures. In any case, latent p53 is tightly associated with Parc in the cytoplasm of unstressed cells. However, in response to DNA damage and other types of stress, p53 is rapidly stabilized and translocated into the nucleus while no significant effect on Parc subcellular localization was observed (see FIGS. 11A-11C, 12A and 12B). As such, regulation of the Parc-p53 interaction in response to stress is an extremely important issue that warrants further investigation. Since p53 is subjected to post-translational modifications in stressed cells, it is possible that phosphorylation and/or acetylation of the p53 protein may regulate its interaction with Parc.

Subcellular Localization and p53 Degradation

Based on the observations that p53 can be stabilized by blocking the nuclear export (Freedman and Levine, 1998; Stommel et al., 1999), several earlier studies proposed that additional cytoplasmic factors may be required for complete degradation of p53 in the cytoplasm although more recent studies reported that degradation of p53 can also be carried out, to some extent, by nuclear proteasomes (Xirodimas et al., 2001). Although Mdm2 is a potent ubiquitin ligase, it was reported that Mdm2 more efficiently induces monoubiquitination, but not polyubiquitination of p53 (Honda et al., 1997; Lai et al., 2001). Since polyubiquitination is generally required for proteasome-mediated degradation (Pickart, 2001), it is possible that there is another ubiquitin ligase (E3) or multiubiquitin chain assembling enzyme (E4) resides in the cytoplasm and contributes to the efficiency of p53 degradation.

In several respects, the Parc protein is an appropriate candidate for such a factor. Parc can tether p53 in the cytoplasm and like Parkin, it contains a signature ubiquitin ligase motif. Moreover, we have found that Parc has an intrinsic ubiquitin ligase activity and can ubiquitinate itself very efficiently. However, Parc fails to induce direct ubiquitination of p53 to a significant degree in vivo or in vitro. Moreover, ablation of endogenous Parc expression strongly induced p53 nuclear localization but did not significantly affect p53 protein levels. Thus, our study indicates that a primary function of Parc is to control the subcellular localization of p53. We can not, however, exclude the possibility that Parc is also involved in the regulation of p53 ubiquitination in the presence of other cofactors. Very recently, the CHIP protein, which contains a multiubiquitin chain assembling enzyme E4-like activity, was shown to interact functionally and physically with the Ring-IBR-Ring domain of Parkin (Imai et al., 2002). Since Parc also contains the Ring-IBR-Ring domain, it is possible that Parc may recruit the CHIP-mediated E4 activity to induce polyubiquitination of p53 for degradation.

The relationship between Parc- and Mdm2-mediated negative regulations on p53 has not been fully elucidated. Although both proteins are p53-associated ubiquitin ligases, they apparently regulate p53 function through different mechanisms. In fact, Mdm2 also plays a critical role in promoting nuclear export of p53 in addition to ubiquitination-mediated degradation of p53 (Geyer et al., 2000; Boyd et al., 2000). Thus, it is most likely that Parc and Mdm2 cooperatively regulate subcellular localization and stability of p53 and more effectively keep p53 under control.

### REFERENCES

Abou Elela, S., and Nazar, R. N. (1997). The ribosomal 5.8S RNA as a target site for p53 protein in cell differentiation and oncogenesis. Cancer Lett 117, 23-28.

Bosari, S., Viale, G., Roncalli, M., Graziani, D., Borsani, G., Lee, A. K., and Coggi, G. (1995). p53 gene mutations, p53 protein accumulation and compartmentalization in colorectal adenocarcinoma. Am J Pathol 147, 790-798.

Boyd, S. D., Tsai, K. Y., and Jacks, T. (2000). An intact HDM2 RING-finger domain is required for nuclear exclusion of p53. Nat Cell Biol 2, 563-568.

Dignam, J. D., Lebovitz, R. M., and Roeder, R. G. (1983). Accurate transcription initiation by RNA polymerase II in a soluble extract from isolated mammalian nuclei. Nucleic Acids Res 11, 1475-1489.

Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498.

El-Deiry, W. S., Kern, S. E., Pietenpol, J. A., Kinzler, K. W., and Vogelstein, B. (1992). Definition of a consensus binding site for p53. Nature Genet. 1, 45-49.

Fontoura, B. M., Atienza, C. A., Sorokina, E. A., Morimoto, T., and Carroll, R. B. (1997). Cytoplasmic p53 polypeptide is associated with ribosomes. Mol Cell Biol 17, 3146-3154.

Freedman, D. A., and Levine, A. J. (1998). Nuclear export is required for degradation of endogenous p53 by MDM2 and human papillomavirus E6. Mol Cell Biol 18, 7288-7293.

Gannon, J. V., and Lane, D. P. (1991). Protein synthesis required to anchor a mutant p53 protein which is temperature-sensitive for nuclear transport. Nature 349, 802-806.

Geyer, R. K., Yu, Z. K., and Maki, C. G. (2000). The MDM2 RING-finger domain is required to promote p53 nuclear export. Nat Cell Biol 2, 569-573.

Giannakakou, P., Sackett, D. L., Ward, Y., Webster, K. R., Blagosklonny, M. V., and Fojo, T. (2000). p53 is associated with cellular microtubules and is transported to the nucleus by dynein. Nat Cell Biol 2, 709-717.

Gu, W., Malik, S., Ito, M., Yuan, C. X., Fondell, J. D., Zhang, X., Martinez, E., Qin, J., Roeder, R. G. (1999). A novel human SRB/MED-containing cofactor complex, SMCC, involved in transcription regulation. Mole. Cell 3, 97-108.

Gu, J., Nie, L., Wiederschain, D., and Yuan, Z-M. (2001). Identification of p53 sequence elements that are required for Mdm2-mediated nuclear export. Mol. Cell. Biol. 21, 8533-8546.

Guo, A., Salomoni, P., Luo, J., Shih, A., Zhong, S., Gu, W., Pandolfi, P. P. (2000). The function of PML in p53-dependent apoptosis. Nature Cell Biol. 2, 730-736

Hollstein, M., Hergenhahn, M., Yang, Q., Bartsch, H., Wang, Z. Q., Hainaut, P. (1999). New approaches to understanding p53 gene tumor mutation spectra. Mutat Res 431,199-209.

Honda, R., Tanaka, H. & Yasuda, H. Oncoprotein MDM2 is a ubiquitin ligase E3 for tumor suppressor p53. FEBS Lett. 420, 25-27 (1997).

Imai, Y., Soda, M., Hatakeyama, S., Akagi, T., Hashikawa, T., Nakayama, K. I., and Takahashi, R. (2002). CHIP is associated with Parkin, a gene responsible for familial Parkinson's disease, and enhances its ubiquitin ligase activity. Mol Cell 10, 55-67.

Jimenez, G. S., Khan, S. H., Stommel, J. M., and Wahl, G. M. (1999). p53 regulation by post-translational modification and nuclear retention in response to diverse stresses. Oncogene 18, 7656-7665.

Kitada, T., Asakawa, S., Hattori, N., Matsumine, H., Yamamura, Y., Minoshima, S., Yokochi, M., Mizuno, Y., and Shimizu, N. (1998). Mutations in the parkin gene cause autosomal recessive juvenile parkinsonism. Nature 392, 605-608.

Klotzsche, O., Etzrodt, D., Hohenberg, H., Bohn, W., and Deppert, W. (1998). Cytoplasmic retention of mutant p53 is dependent on an intermediate filament protein (vimentin) scaffold. Oncogene 16, 3423-3434.

Lai Z., Ferry, K. V., Diamond, M. A., Wee K. E., Kim, Y. B., Ma, J., Yang, T., Benfield, P. A., Copeland, R. A., Auger, K. R. (2001) Human mdm2 mediates multiple monoubiquitination of p53 by a mechanism requiring enzyme isomerization. J Biol Chem. 276, 31357-67.

Li, M., Chen, D., Shiloh, A., Luo, J., Nikolaev, A. Y., Qin, J., and Gu, W. (2002a). Deubiquitination of p53 by HAUSP is an important pathway for p53 stabilization. Nature 416, 648-653.

Li, M., Luo, J., Brooks, C. L., and Gu. W. (2002b) Acetylation of p53 inhibits its ubiquitination by Mdm2. J. Biol. Chem. published on line, Nov. 5, 2002 as 10.1074/jbc.C200578200.

Liang, S. H., Hong, D., and Clarke, M. F. (1998). Cooperation of a single lysine mutation and a C-terminal domain in the cytoplasmic sequestration of the p53 protein. J Biol Chem 273, 19817-19821.

Lohrum, M. A., Woods, D. B., Ludwig, R. L., Balint, E., and Vousden, K. H. (2001). C-terminal ubiquitination of p53 contributes to nuclear export. Mol Cell Biol 21, 8521-8532.

Luo, J., Su, F., Chen, D., Shiloh, A., Gu, W. (2000). Deacetylation of p53 modulates its effect on cell growth and apoptosis. Nature 408, 377-381.

Luo, J., Nikolaev, A. Y., Imai, S., Chen, D., Su, F., Shiloh, A., Guarente, L., and Gu, W. (2001). Negative control of p53 by Sir2alpha promotes cell survival under stress. Cell 107, 137-148.

Metcalfe, S., Weeds, A., Okorokov, A. L., Milner, J., Cockman, M., and Pope, B. (1999). Wild-type p53 protein shows calcium-dependent binding to F-actin. Oncogene 18, 2351-2355.

Middeler, G., Zerf, K., Jenovai, S., Thulig, A., Tschodrich-Rotter, M., Kubitscheck, U., and Peters, R. (1997). The tumor suppressor p53 is subject to both nuclear import and export, and both are fast, energy-dependent and lectin-inhibited. Oncogene 14, 1407-1417.

Moll, U. M., LaQuaglia, M., Benard, J., and Riou, G. (1995). Wild-type p53 protein undergoes cytoplasmic sequestration in undifferentiated neuroblastomas but not in differentiated tumors. Proc Natl Acad Sci USA 92, 4407-4411.

Moll, U. M., Ostermeyer, A. G., Haladay, R., Winkfield, B., Frazier, M., and Zambetti, G. (1996). Cytoplasmic sequestration of wild-type p53 protein impairs the G1 checkpoint after DNA damage. Mol Cell Biol 16, 11261137.

Moll, U. M., Riou, G., and Levine, A. J. (1992). Two distinct mechanisms alter p53 in breast cancer: mutation and nuclear exclusion. Proc Natl Acad Sci USA 89, 7262-7266.

Pickart, C. M. (2001). Mechanisms underlying ubiquitination. Annu Rev Biochem 70, 503-533.

Schlamp, C. L., Poulsen, C. L., Nork, T. M., and Nickells, R. W. (1997). Nuclear exclusion of wild-type p53 in immortalized human retinoblastoma cells. J Natl Cancer Inst 89, 1530-1536.

Sengupta, S., Vonesch, J. L., Waltzinger, C., Zheng, H., and Wasylyk, B. (2000). Negative cross-talk between p53 and the glucocorticoid receptor and its role in neuroblastoma cells. EMBO J. 19, 6051-6064.

Sharpless, N. E., and DePinho, R. A. (2002). p53. Good Cop/Bad Cop. Cell 110, 9-12.

Shaulsky, G., Goldfinger, N., Ben-Ze'ev, A., and Rotter, V. (1990). Nuclear accumulation of p53 protein is mediated by several nuclear localization signals and plays a role in tumorigenesis. Mol Cell Biol 10, 6565-6577.

Shimura, H., Hattori, N., Kubo, S., Mizuno, Y., Asakawa, S., Minoshima, S., Shimizu, N., Iwai, K., Chiba, T., Tanaka, K., and Suzuki, T. (2000). Familial Parkinson disease gene product, parkin, is a ubiquitin-protein ligase. Nat Genet 25, 302-305.

Stommel, J. M., Marchenko, N. D., Jimenez, G. S., Moll, U. M., Hope, T. J., and Wahl, G. M. (1999). A leucine-rich nuclear export signal in the p53 tetramerization domain: regulation of subcellular localization and p53 activity by NES masking. Embo J 18, 1660-1672.

Sun, X. F., Carstensen, J. M., Zhang, H., Stal, O., Wingren, S., Hatschek, T., and Nordenskjold, B. (1992). Prognostic significance of cytoplasmic p53 oncoprotein in colorectal adenocarcinoma. Lancet 340, 1369-1373.

Tao, W., Levine, A. J. (1999). Nucleocytoplasmic shuttling of oncoprotein Hdm2 is required for Hdm2-mediated degradation of p53. Proc Natl Acad Sci USA 96, 3077-3080.

Ueda, H., Ullrich, S. J., Gangemi, J. D., Kappel, C. A., Ngo, L., Feitelson, M. A., and Jay, G. (1995). Functional inactivation but not structural mutation of p53 causes liver cancer. Nat Genet 9, 41-47.

Vogelstein, B., Lane, D., and Levine, A. J. (2000). Surfing the p53 network. Nature 408, 307-310.

Vousden, K. H. (2002). Activation of the p53 tumor suppressor protein. Biochim Biophys Acta 1602, 47-59.

Xirodimas, D. P., Stephen, C. W., and Lane, D. P. (2001). Cocompartmentalization of p53 and Mdm2 is a major determinant for Mdm2-mediated degradation of p53. Exp Cell Res 270, 66-77.

Zaika, A., Marchenko, N., and Moll, U. M. (1999). Cytoplasmically “sequestered” wild type p53 protein is resistant to Mdm2-mediated degradation. J Biol Chem 274, 27474-27480.

Zhang, Y., and Xiong, Y. (2001). A p53 amino-terminal nuclear export signal inhibited by DNA damage-induced phosphorylation. Science 292, 1910-1915.

What is claimed is:

1. An isolated p53-associated Parkin-like cytoplasmic protein (Parc).

2. The protein of claim 1, wherein the protein is a human protein.

3. The protein of claim 2, wherein the protein comprises the amino acid sequence set forth in FIG. 14.

4. A recombinant protein comprising the N-terminal 770 residues of the amino acid sequence set forth in FIG. 14.

5. The protein of claim 4 consisting of the N-terminal 770 residues of the amino acid sequence set forth in FIG. 14.

6. An isolated nucleic acid encoding the protein of claim 1.

7. An isolated nucleic acid encoding the protein of claim 2.

8. An isolated nucleic acid encoding the protein of claim 3.

9. An isolated nucleic acid encoding the protein of claim 4.

10. The nucleic acid of any of claims 6-9, wherein the nucleic acid is DNA.

11. The nucleic acid of any of claims 6-9, wherein the nucleic acid is RNA.

12. An expression vector comprising the nucleic acid of any of claims 6-9.

13. A host vector system comprising a cell having therein the expression vector of claim 12.

14. A method for producing a protein comprising culturing the host vector system of claim 13 under conditions permitting the expression of the protein encoded by the expression vector therein, and recovering the protein so expressed.

15. A nucleic acid which hybridizes to at least a portion of an RNA encoding Parc.

16. The nucleic acid of claim 15, wherein the Parc is human Parc.

17. The nucleic acid of claim 15, wherein the Parc comprises the amino acid sequence set forth in FIG. 14.

18. The nucleic acid of claim 17, wherein the nucleic acid hybridizes to at least a portion of RNA encoding the N-terminal 770 amino acid residues set forth in FIG. 14.

19. An antibody which binds to the protein of claim 1.

20. An antibody which binds to the protein of claim 2.

21. An antibody which binds to the protein of claim 3.

22. An antibody which binds to the protein of claim 5.

23. The antibody of any of claims 19-22, wherein the antibody is detectably labeled.

24. The antibody of any of claims 19-22, wherein the antibody is immobilized.

25. A method for making an antibody which binds to Parc comprising the steps of:
(a) introducing Parc to a mammal under conditions which permit the generation of antibodies to an antigen; and
(b) after a suitable period of time, recovering antibodies generated in the mammal which bind to Parc.

26. A method for determining whether an agent inhibits binding between p53 and Parc comprising
(a) contacting the agent with p53 and Parc under conditions which, in the absence of the agent, permit the formation of a complex between p53 and Parc;
(b) determining the amount of complex formed in step (a) between p53 and Parc; and
(c) comparing the amount of complex determined in step (b) with the amount of complex formed in the absence of the agent, wherein if the amount of complex formed in step (a) is less than the amount formed in the absence of the agent, the agent inhibits binding between p53 and Parc.

27. The method of claim 26, wherein the p53 and Parc are human p53 and human Parc.

28. A method for determining whether an agent inhibits binding between p53 and the p53-binding portion of Parc comprising
(a) contacting the agent with p53 and the p53-binding portion of Parc under conditions which, in the absence of the agent, permit the formation of a complex between p53 and the p53-binding portion of Parc;
(b) determining the amount of complex formed in step (a) between p53 and the p53-binding portion of Parc; and
(c) comparing the amount of complex determined in step (b) with the amount of complex formed in the absence of the agent, wherein if the amount of complex formed in step (a) is less than the amount formed in the absence of the agent, the agent inhibits binding between p53 and the p53-binding portion of Parc.

29. The method of claim 28, wherein the p53 and p53-binding portion of Parc are human p53 and the p53-binding portion of human Parc.

30. The method of claim 28, wherein the p53-binding portion of Parc comprises the N-terminal 770 amino acid residues of Parc set forth in FIG. 14.

31. A method for determining whether an agent inhibits binding between p53 and Parc in a cell comprising
(a) contacting the agent with the cell under suitable conditions;
(b) determining the amount of complex formed in the cell between Parc and p53; and
(c) comparing the amount of complex determined in step (b) with the amount of complex formed in the absence of the agent, wherein if the amount of complex formed in step (a) is less than the amount formed in the absence of the agent, the agent inhibits binding between p53 and Parc in the cell.

32. The method of claim 31, wherein the cell is a human cell.

33. The method of claim 32, wherein the cell is selected from the group consisting of a neuroblastoma cell, a breast cancer cell, a colorectal cancer cell, and a retinoblastoma cell.

34. A method for determining whether an agent inhibits the expression of Parc in a cell comprising
(a) contacting the agent with the cell under suitable conditions;
(b) determining the amount of Parc expressed in the cell; and
(c) comparing the amount of Parc expression determined in step (b) with the amount of Parc expression in the cell in the absence of the agent, wherein if the amount of Parc expression determined in step (b) is less than the amount of Parc expression in the absence of the agent, the agent inhibits expression of Parc in the cell.

35. The method of claim 34, wherein the cell is a human cell.

36. The method of claim 35, wherein the cell is selected from the group consisting of a neuroblastoma cell, a breast cancer cell, a colorectal cancer cell, and a retinoblastoma cell.

37. The method of claim 34, wherein determining the amount of Parc expression in the cell is performed via determining the amount of Parc present in the cell.

38. The method of claim 34, wherein determining the amount of Parc expression in the cell is performed via determining the amount of Parc-encoding mRNA in the cell.

39. A method for decreasing the amount of Parc in a cell comprising introducing into the cell an agent that specifically inhibits the expression of Parc in the cell, thereby decreasing the amount of Parc in the cell.

40. The method of claim 39, wherein the cell is a human cell.

41. The method of claim 40, wherein the cell is selected from the group consisting of a neuroblastoma cell, a breast cancer cell, a colorectal cancer cell, and a retinoblastoma cell.

42. The method of claim 39, wherein the agent is an antisense RNA which hybridizes to Parc-encoding mRNA.

43. The method of claim 39, wherein the agent is a ribozyme which hybridizes to Parc-encoding mRNA.

44. The method of claim 39, wherein the agent is a DNAzyme which hybridizes to Parc-encoding mRNA.

45. The method of claim 40, wherein the cell's p53 is wild-type p53.

46. A method for treating a subject afflicted with cancer comprising administering to the subject a therapeutically effective amount of an agent that specifically inhibits the expression of Parc in the subject's cells, thereby treating the subject.

47. The method of claim 46, wherein the subject is human.

48. The method of claim 47, wherein the subject's cells comprise wild-type p53.

49. The method of claim 47, wherein the cancer is a neuroblastoma, breast cancer, colorectal cancer, or a retinoblastoma.

50. The method of claim 46, wherein the agent is an antisense RNA, a ribozyme, or a DNAzyme which hybridizes to Parc-encoding mRNA.

51. The method of claim 50, wherein the subject is human and the antisense RNA, ribozyme, or DNAzyme hybridizes to the portion of the mRNA which encodes the N-terminal 770 amino acid residues of Parc.

52. A composition comprising a pharmaceutically acceptable carrier and an agent that specifically inhibits the expression of Parc in a cell.

53. The composition of claim 52, wherein the agent is an antisense RNA which hybridizes to Parc-encoding mRNA.

54. The composition of claim 52, wherein the agent is a ribozyme which hybridizes to Parc-encoding mRNA.

55. The composition of claim 52, wherein the agent is a DNAzyme which hybridizes to Parc-encoding mRNA.

56. A method for determining whether a cell is cancerous comprising determining the amount of Parc in the cell and comparing the amount so determined to the amount of Parc present in a cell either known to be cancerous or known not to be cancerous, thereby determining whether the cell is cancerous.

57. The method of claim 56, wherein the cell is human.

58. The method of claim 57, wherein the cell is obtained from a subject suspected of having a neuroblastoma, breast cancer, colorectal cancer, or a retinoblastoma.

59. A kit for determining the amount of Parc present in a sample comprising a detectably labeled agent which binds to Parc, and instructions for use.

60. The kit of claim 59, wherein the agent is an antibody.

61. The kit of claim 60, wherein the antibody is immobilized.

62. The kit of claim 59, wherein the Parc is human Parc.

63. A kit for determining the amount of Parc-encoding nucleic acid present in a sample comprising a nucleic acid capable of hybridizing to Parc-encoding nucleic acid, and instructions for use.

64. The kit of claim 63, wherein the nucleic acid is immobilized.

65. The kit of claim 63, wherein the Parc is human Parc.
